SEQUENCE LISTING (2 sequences)

SEQ ID NO: 1 - protein, 511 aa, Vesicular stomatitis virus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 1447 aa, Human immunodeficiency virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHIARNCRAPRKKGCWKCGKEGHQMKDCTERQANFLREDLAFLQGKAREFSSEQTRANSPTISSEQTRANSPTRRELQVWGRDNNSLSEAGADRQGTVSFNFPQITLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARTRGAHTNDVKQLTEAVQKITTESIVIWGKTPKFKLPIQKETWETWWTEYWQATWIPEWEFVNTPPLVKLWYQLEKEPIVGAETFYVDGAASRETKLGKAGYVTNRGRQKVVTLTDTTNQKTELQAIHLALQDSGLEVNIVTDSQYALGIIQAQPDKSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTSTTVKAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRDPLWKGPAKLLWKGEGAVVIQDNSDIKVVPRRKAKIIRDYGKQMAGDDCVASRQDED

(19) United States
(12) Patent Application Publication
(10) Pub. No.: US 2018/0127470 A1
(43) Pub. Date: May 10, 2018

(54) Cell Lines

(71) Applicant: OXFORD GENETICS LTD, Oxford, Oxfordshire (GB)

(72) Inventor: Ryan Cawood, Oxford, Oxfordshire (GB)

(21) Appl. No.: 15/576,384

(22) PCT Filed: May 27, 2016
(86) PCT No.: PCT/GB2016/051554

§ 371 Date: Nov. 22, 2017

(30) Foreign Application Priority Data
May 27, 2015 (GB) 1509040.0

(51) Int. Cl. C07K 14/005; C12N 15/86; C07K 14/47; C12N 7/00

(52) U.S. Cl. CPC C07K 14/005; C12N 15/86; C07K 14/4747; C12N 7/00; C12N 2840/203; C12N 2740/16311; C12N 2740/16211; C12N 2760/20252; C12N 2710/10352; C12N 2740/15052; C12N 2810/6081

(57) ABSTRACT

The present invention relates to a process for producing a cell which constitutively expresses cytotoxic virus poly-peptides (e.g. VSV G or Gag-Pol). The invention also provides plasmids/vectors and kits for use in the production of the cells. Furthermore, the invention provides a process for producing retroviruses using the cells of the invention.

The present invention relates to a process for producing a cell which constitutively expresses cytotoxic virus polypeptides (e.g. VSV G or Gag-Pol). The invention also provides plasmids/vectors and kits for use in the production of the cells. Furthermore, the invention provides a process for producing retroviruses using the cells of the invention.

Retroviruses (including lentiviruses) are positive sense RNA viruses that undergo a complex life cycle involving the reverse transcription of their genome into deoxyribonucleic acid (DNA), which subsequently becomes integrated into the host cell genome following viral infection. They are capable of inserting their genomes, as DNA, into almost any loci in the genome of target cells and mediating long term expression of virus genes, with the DNA being copied into each daughter cell when the infected cell divides. They generate their genome as an un-spliced mRNA molecule by using the cellular RNA polymerase for transcription. The virus genome is then transported into the cytoplasm using a virus protein called Rev. The genome is then packaged into virus particles in the cytosol using the virus encoded structural proteins Envelope (env), Gag and Polymerase (Pol). The retrovirus genome is typically 7-10 kb in length, in the case of the commonly studied HIV virus the genome is 9.7 kb in length. It exists in each virus particle at 2 copies per virion.

The retrovirus life cycle, and their structural flexibility, affords a number of biotechnological applications, such as the delivery of DNA into the genome of mammalian cells. Further, retroviruses can be modified to contain non-retrovirus glycoproteins in their surface, endowing retrovirus particles with the cellular tropism of the virus from which the glycoprotein originated. This is particularly important when the natural retrovirus glycoprotein has a limited cellular tropism. An example of this is the GP160 glycoprotein of HIV-1, which has evolved to bind the CD4 receptor and only infects cells bearing this protein on their surface. In the case of HIV-1, virus particles are frequently modified to contain a glycoprotein that is different from the natural glycoprotein in a process called pseudotyping. Most commonly this is achieved with the glycoprotein from vesicular stomatitis virus (VSV G) to provide a much broader cell tropism.

When using retroviruses in the laboratory as tools, they are typically modified to form replication-incompetent vectors that can express either one or more transgenes or shRNA molecules, and these modified viruses provide versatile vectors for cellular transgene expression and engineering. The flexibility of the retrovirus packaging process also allows for varying genome sizes to be accommodated: genomes as small as 3 kb and as large as 18 kb can be packaged, although virus titre can be compromised at these extremes.

Several clinical trials have now been performed, using retroviruses (and latterly lentiviruses) to infect stem cells ex vivo to express transgenes to be supplemented in the treatment of inherited single gene disorders, before reintroducing them into patients. This is usually done on an autologous basis, although some stem cells can also be applied as heterologous transplants. Retro/lentiviruses are also finding important applications in the field of adoptive cell transfer, most notably to allow expression of hybrid ‘chimeric antigen receptors’ (CAR) within T-cells before cell expansion, and reinfusion into patients. The CARs generally have an extracellular antibody structure, and an intracellular structure based on the T-cell receptor but modified (in 2nd and 3rd generation CARs) to improve the quality of cell stimulation following binding of the outside portion to its antigen. This ‘CAR T cell’ approach has shown impressive success using lentiviruses encoding CARs recognising CD19 in the clinical treatment of B cell lymphoma, and the first US product licence is expected to be granted to Novartis for their CD19-specific CART cell, known as CTL019, in the near future. The field of application is now being expanded to address other molecular targets and other malignancies. Hence, there is an expanding need for large scale lentivirus manufacture, something that is challenging to achieve using existing virus production systems. Alongside clinical use, many laboratories frequently use lentivirus vectors for research and development, where the insertion of exogenous DNA into the cellular genome is required. The versatility of lentiviruses has allowed them to be used to introduce DNA into a wide range of cell types, including but not limited to, human and mouse stem cells, cancer cells, primary tissue cells (e.g. liver, neurons, fibroblasts). The infection of these cells is only made possible by coating, or pseudotyping, the virus with a broad tropism glycoprotein, most commonly the VSV G surface glycoprotein. This protein enables the infection of cells from almost all organs and across many species, including but not limited to, humans, mice, rats, hamsters, monkeys, rabbits, donkeys and horses, sheep, cows and old world apes.

Although wild type retro/lentiviruses can replicate in host cells, the retro/lentivirus vectors used for transgene and shRNA expression are typically disabled in a range of ways to remove their ability to replicate and cause disease. This means that in order to grow a batch of infectious virus particles, capable of a single infection round, for experimental or clinical use, it is necessary to provide several virus genes (and thereby virus proteins) that have been genetically removed from the virus genome at the same time into the cells used for virus packaging. These genes are generally provided in three or four separate plasmids, co-transfected into cells. The central component is a plasmid encoding the virus vector genome (including any transgenes and associated promoters to regulate transcription in target cells) containing packaging signals to direct the assembling virus particles to incorporate the corresponding RNA into the new virus particles. Virus ancillary proteins such as Gag-Pol, Tat and Rev genes are generally provided from other plasmids that are co-transfected, and yet another plasmid provides the glycoprotein to be incorporated into the envelope of newly formed virus particles, that will direct their infectious tropism. The gag-pol expression cassette encodes virus capsid and internal structural proteins and polymerase and protease activity. The Rev gene acts to enhance nuclear export of retro/lentivirus genomes by binding to a specific region of the virus genome called the Rev Response Element (RRE).

The complexity of retrovirus and lentivirus packaging systems has resulted in a number of ‘generations’, each with increasing safety on the previous system. In the ‘1st generation’ packaging systems, three plasmids were used: one plasmid encoding all of the HIV genes except for the Envelope gene; a second plasmid to provide a surface glycoprotein (most often VSV G); and a plasmid containing the virus genome to be packaged. This system has the disadvantage that the plasmid containing the virus genes contained large regions of DNA with homology to the virus genome plasmid, potentially allowing for recombination between plasmids. This could result in infectious virus being produced capable of causing disease. Other problems included the presence of many virus genes that were not needed for the virus production, including VPU, VIF, VPR and Nef.

In the ‘2nd generation’ systems, five of the nine HIV 1 gene coding regions were removed from the system. This method also resulted in a three plasmid system, with one plasmid containing the Gag-Pol genes and the ancillary genes for Tat and Rev proteins, a second plasmid encoding a glycoprotein (most often VSV G) and a third plasmid that encoded the virus genome to be packaged. The virus genomes in this system typically contain a wild type 5′ Long Terminal Repeats (LTR) and hence require the Tat gene for transcriptional activation and genome production. This system had the advantage that the reduction in homology between the virus genome and the packaging plasmids reduced the likelihood of the formation of potentially hazardous replication competent retrovirus.

In the most recent ‘3rd generation’ lentiviral vector system, four plasmids are used instead of three. By splitting the system into 4 plasmids (3 helper plasmids and 1 containing the vector genome plus transgene), the ‘3rd generation’ system offers a number of advantages (primarily by increasing the number of recombination events required to form replication-competent virus). However, the ‘3rd generation’ systems also have another significant advantage because they have a modified 5′LTR that includes a promoter, and hence transcription of the genome is not dependent on transcriptional activation by the Tat protein—thereby removing the need for Tat to be encoded in the system. They do not contain the Tat protein on any of the plasmids used. The Rev gene was also placed on an individual plasmid. Therefore, in 3rd generation systems, the four plasmids contain 1: Gag-Pol, 2: a glycoprotein (most frequently VSV G), 3: Rev, and 4: a plasmid encoding a self-inactivating lentivirus genome containing the transgene or RNA of interest. With specific reference to the glycoprotein plasmid, several envelope glycoproteins are available and have been used, but the most widely used is the glycoprotein from Vesicular Stomatitis Virus, known as VSV G.

Some of these lentivirus packaging genes, notably the VSV G and Gag-Pol components, are widely reported to be toxic to mammalian cells (Burns et al., Proc. Natl. Acad. Sci. 90, 8033-8037 (1993); Yee et al., Proc. Natl. Acad. Sci., 90, 9564-9568 (1994); Hoffman et al., J. Gen. Virol, 91, 2782-2793 (2010)). This has provided a substantial barrier to the development of stable packaging cells that express many of the required packaging proteins. Accordingly, batches of lentivirus have been prepared by an inefficient process requiring simultaneous expression of all the plasmids in cells by transient transfection.

Such transfection methods are expensive, hard to reproduce at large scale, and often lead to contamination of the virus preparation with plasmids and cellular debris.

It is highly desirable to create ‘packaging’ cell lines for retroviruses and lentiviruses that encode some, or all, of the components required for production of new virus particles within the cellular genome. This could decrease the complexity of the plasmid transfection required for virus packaging and has the major benefit that every cell will be expressing the genes required for virus production. The ability to create cell lines that express virus proteins with a specific stoichiometry relative to each other would be another significant advantage. There have been several attempts to express virus proteins either stably or under conditional or inducible promoters, for example the STAR cells produced by Ikeda et al. (Nature Biotechnology, 21, 560-572 (2003)) used retroviral transduction of codon-optimised HIV Gag, Pol and Rev to achieve continuous expression in packaging cells. However, the titre of virus produced using these cells is typically below the industry standard of 1×10⁷-1×10⁸/ml. The requirement that some genes must also be inducible, or require independent antibiotic selection agents significantly adds to the system's complexity, and makes scaling up for manufacture significantly more challenging. To date, there have been no cell lines produced that stably and constitutively express the most commonly used retrovirus and lentivirus glycoprotein VSV G due to its reported toxicity.

One component that would be highly desirable to express in high amounts and constitutively is the VSV G envelope glycoprotein, which mediates a broad infectious tropism. VSV G protein is a single pass membrane glycoprotein derived from the Vesicular Stomatitis virus. The gene is encoded by a 1536 bp open reading frame and produces a protein consisting of 511 amino acids. The protein contains a 16 amino signal peptide at the N-terminus (amino acid sequence: MLSYLIFALAVSPILG) which is cleaved from the mature protein during export through the secretory pathway to the cell surface. The glycoprotein contains an extracellular region of 458 amino acids and a membrane spanning region (transmembrane region) of 21 amino acids followed by an intracellular (cytosolic) c-terminal region of 22 amino acids. The shuttling of VSV G protein from the endoplasmic reticulum is rapid, and this is achieved by the specific trafficking signals in the c-terminal tail, including a DxE motif (where x is any amino acid) within the broader trafficking signal Tyr-Thr-Asp-Ile-Glu-Met that contains the DxE motif (Sevier et al., Mol. Biol. Cell. 2000 January; 11(1): 13-22). The efficiency of export of VSV G protein may in part contribute to its effectiveness for retrovirus and lentivirus production. The VSV G receptor is frequently described as a non-specific fusogenic protein, however is was recently determined the VSV G binds to the low-density lipid receptor (LDL-R) (Finkelstein et al., Proc. Natl. Acad. Sci. USA 2013; 110(18):7306-7311), which explains its broad cellular tropism and broad application in retrovirus and lentivirus pseudotyping.

A packaging cell line that stably expresses VSV G would be useful to the production of all types of retrovirus and lentivirus requiring a VSV G envelope. However, it is widely accepted that constitutive expression of VSV G in cells is toxic (Burns et al., Proc. Natl. Acad. Sci. 90, 8033-8037 (1993); Yee et al., Proc. Natl. Acad. Sci., 90, 9564-9568 (1994); Hoffman et al., J. Gen. Virol, 91, 2782-2793 (2010), and that the creation of a stably-expressing VSV G cell line is extremely challenging and perhaps impossible. For example, in their 2015 attempt to produce a stable lentivirus packaging cell, Sanber et al. (Scientific Reports, 5, 9021) chose to avoid use of VSV G entirely because of its cytotoxicity, despite its acknowledged broad utility.

Another component that would be highly desirable to express in high amounts and constitutively is the Gag-Pol protein. The Gag-Pol protein of lentiviruses is produced as a single poly-protein that encodes a protease that enables the proteolytic cleavage of the Gag-Pol protein into a number of smaller proteins serving a number of virus functions. The HIV-1 Gag protein is produced from the first translated open reading from the 5′ end of the virus genome and contains a sequence known as the frame-shift sequence. This signal causes the translating ribosome to shift back on the mRNA molecule one base during translation approximately every 1 in 20 translation runs. This process produces the Gag-Pol protein. The result is that lentivirus produce Gag and Gag-Pol at an approximate ratio of 1:20. The Gag protein encodes three major structural proteins: p18, p24 and p15. The Pol protein segment also encodes three major proteins called p10 (protease), p66/55 (reverse transcriptase) and p32 (integrase). The protease is responsible for all of the cleavage events required to produce each of these proteins by proteolytic cleavage. However, the protease recognition sequences that define these cleavage events are poorly defined, suggesting that the protease has broad specificity. This is therefore likely to result in the cleavage of proteins that are not virus related. Indeed, the expression of Gag-Pol proteins is reported to be highly toxic to cells because of this (Blanco et al., The Journal of Biochemistry, 278, 2, 1086-1093, 2003).

It has now been found that the toxicity associated with the expression of cytotoxic virus proteins such as VSV G and Gag-Pol protein in cell lines may be mitigated or prevented by co-expressing one or more apoptosis inhibitor(s) in the cells.

Apoptosis is the cellular process of programmed cell death. It can be induced under a wide range of physiological settings that activate a series of cellular signalling pathways resulting in cells inducing their own destruction. The apoptosis biochemical pathways can be induced from signals that are both external (extrinsic) and internal (intrinsic) to the cell. For example, the binding of TNF-Alpha or FAS-ligand to the outer surface receptors of some cell types can rapidly induce apoptosis. These are considered examples of extrinsic signals. Examples of intrinsic signals would include the induction of apoptosis in response to extensive DNA damage, virus infection (RIG and NFKB signalling), or the loss of membrane integrity (which can be measured by intra-cellular sensors as an increase in calcium concentrations using the cell sensor Calpain). These are considered intrinsic signals to initiate apoptosis. Regardless of the method of induction, the final stages of apoptosis are shared, and can be confirmed morphologically. These initially include nuclear condensation (termed pyknosis) and cell shrinkage, coupled with ‘blebbingx’ which is the bulging and protrusion of the cells outer membrane. These early phases are followed by nuclear fragmentation (karyorrhexis) and the formation of apoptotic bodies (fragments of the dead cell).

Given that apoptosis is a controlled protein- and enzyme-driven process, some proteins can increase or decrease a cell's ability to control and induce the apoptosis process. Indeed, many proteins have been discovered that can induce apoptosis (such as BAX, BAD, BAK and BOK); others have been found to inhibit apoptosis (such as human AVEN, human Bcl-2, Adenovirus E1B-19K and a range of other viral proteins).

Some apoptosis-inhibiting genes have previously been used to improve the viability of cells used for protein manufacture, for example, US 2010/0167396 A1 (Murphy et al.). However, this latter patent application relates specifically to the production of Factor VIII (which is a non-toxic protein), wherein the anti-apoptosis polypeptides are used merely to prevent or delay apoptosis in order to facilitate an increased production of Factor VIII.

US 2003/0064510 (Reff et al.) relates to the use of the apoptosis inhibitors AVEN and E1B-19K for the general prevention of apoptosis in cell lines which are intended to be used to improve the production of native or heterologous proteins or viruses. The product targets are said to include antibodies, cytokines, growth factors, hormones, serum proteins, receptors, enzymes, ligands, cell secretory factors, cell metabolites and viral vectors. However, as with US 2010/0167396 A1, the aim is merely to increase production of the desired products. In both of these US patent applications, the aim is to delay or prevent apoptosis, wherein apoptosis is considered merely to be a normal part of the physical process of producing recombinant proteins. Neither of these patent applications relates to the production of biologically-cytotoxic proteins; and neither of these patent applications aims to produce stable cell lines expressing virus proteins to allow more efficient production of recombinant retroviruses.

It is therefore an object of the current invention to provide a process for producing stable cell lines that constitutively express one or more cytotoxic virus polypeptides, e.g. VSV G and/or Gag-Pol.

This process facilitates the production of recombinant retroviruses by improving the health of cells expressing cytotoxic virus polypeptides and allowing them to survive the production process, thereby generating more virus than equivalent cells not expressing the apoptosis inhibitor(s).

The expression of the one or more cytotoxic virus polypeptides is linked to the expression of a selection gene and they are transcribed in the same primary transcript.

In a particularly preferred embodiment of the invention, the selection gene is inserted after (3′) an IRES downstream (3′) of the last stop codon of the nucleic acid encoding one or more cytotoxic virus polypeptides. This provides a configuration where a promoter initiating transcription is upstream (5′) to the coding sequences of the one or more cytotoxic virus polypeptide gene(s) which is then followed (3′) by an IRES which is then followed (3′) by the coding region for selection/resistance gene allowing for cell selection (preferably using Puro). In this configuration, both the one or more cytotoxic virus polypeptide and selectable marker are encoded by the same mRNA, but due to the relatively low efficiency of IRES-mediated translation the one or more cytotoxic virus polypeptides will be translated in greater abundance than the selectable marker. To maintain a selectable phenotype, this will ensure that expression of the one or more cytotoxic virus polypeptides continues at a high level and cannot be silenced.

In one embodiment, the invention provides a process for producing a mammalian cell which constitutively expresses one or more cytotoxic virus polypeptides, the process comprising the steps:
(i) introducing one or more nucleic acid molecules encoding
   (a) one or more cytotoxic virus polypeptides, and
   (b) one or more apoptosis inhibitors into a mammalian cell; and
(ii) culturing the cell under conditions such that the one or more cytotoxic virus polypeptides and the one or more apoptosis inhibitors are constitutively expressed,
   wherein the expression of the one or more apoptosis inhibitors prevents apoptosis of the cell.

In a particularly preferred embodiment, the one or more nucleic acid molecules additionally comprises a selection gene which is located in the same primary transcript as the nucleic acid encoding the one or more cytotoxic virus polypeptides and is transcribed in the same mRNA molecule.

Preferably, the selection gene is inserted after an internal ribosome entry site (IRES) downstream of the stop codon of the nucleic acid encoding the one or more cytotoxic virus polypeptides.

Preferably, one or more of the cytotoxic virus polypeptides are selected from the group consisting of VSV G and Gag-Pol. In some embodiments, the one or more cytotoxic virus polypeptides comprise VSV G and Gag-Pol.

The following terms are used herein:

Gene, coding sequence or open reading frame (ORF)—Any sequence of DNA nucleotides that encodes an RNA, mRNA, non-coding RNA, short hairpin RNA or protein coding RNA.
Expression cassette—A combination of DNA sequences that enables a gene, mRNA or protein to be produced within a cell. Typically an expression cassette will contain a promoter, a coding sequence (gene) and an RNA polymerase termination sequence.
Restriction site or restriction enzyme binding site—A region of DNA that is bound by an endonuclease restriction enzyme, typically, but not limited to, 4-8 nucleotides in length where said binding enables the restriction enzyme to cleave the DNA strand.
Restriction enzyme—A polypeptide that when folded produces a catalytic enzyme that can recognise and bind to a specific sequence within a DNA molecule and cleave the same DNA molecule at the restriction enzyme binding site
DNA—Deoxyribonucleic acid
RNA—Ribonucleic acid
Nucleic acids—Polymeric macromolecules made from nucleotide monomers. In DNA, the purine bases are adenine and guanine, while the pyrimidines are thymine and cytosine. In RNA, the Thymine bases are replaced by uracil.
Untranslated region or UTR—The region of an mRNA molecule that does not encode a protein polypeptide that is either upstream (5′) or downstream (3′) of the start codon of the protein coding sequence. The sequence of the 5′ UTR in DNA is between the transcription initiation point and the start codon of a gene whilst the 3′ UTR is the region between the stop codon and the RNA polymerase termination sequence.
Nucleotide—The structural base unit monomers of a DNA molecule that are composed of a deoxyribose sugar covalently linked at the 5′ to a phosphate group and linked by a glycosidic bond to a base that that may be either a purine or a pyrimidine, typically consisting of either adenine or guanine or either thymine or cytosine, respectively.
Base pair, BP—A pair of nucleotides in separate DNA strands in which the bases of the nucleotides are linked by hydrogen bonding.
Kb—A thousand (1000) bases or nucleotides of DNA.
Promoter or promoter region—Except where discussing the promoter that drives the expression of any gene that is responsible for the origin of replication or bacterial antibiotic resistance selectable marker to function, the promoter region or promoter refers to the promoter of the invention that is designed, and positioned within DNA embodying the invention, to drive the transcription of a gene.
TATA Box or Goldberg Hogness box—The sequence that is upstream of the transcription initiation site of a eukaryotic mRNA that is part of the promoter and recruits transcription factors and transcription initiation factors to the promoter region to initiate transcription.
KOZAK sequence—The ribosomal binding and engagement point of an mRNA in eukaryotic cells, the consensus DNA sequence of which is ACCATGG wherein the start codon of a gene is denoted as the ATG in the same consensus sequence.
Sequences—Any DNA sequence that embodies any part of the invention or any sequence that may reside within an embodiment of the invention.
SEQ ID: The terminology used herein to describe any example DNA sequence that may comprise either a component of the invention, or a complete DNA sequence required to exemplify the invention.
A, T, G, C, R, Y or N when referring to nucleotides—The sequence letter codes are as follows: A=adenine, C=cytosine, G=Guanine, T=Thymine, R=adenine or guanine,
Y=cytosine or Thymine, N=any nucleotide or base.
Plasmid, Vector, Plasmid Vector, Plasmid DNA expression vector, expression vector, DNA vector, DNA plasmid: A circular DNA molecule capable of amplification in a prokaryotic host.
Apoptosis inhibitor or apoptosis inhibiting protein or apoptosis inhibiting RNA—any protein or RNA that prevents or delays the cellular pathways leading to apoptosis and cell death.
Apoptosis—Any biochemical pathway inside a cell that leads to the cell triggering and inducing its own destruction via programmed cell death.
LTR—The long terminal repeat region of a retrovirus
Glycoprotein—A secretory surface protein that is trafficked through the secretory pathway, normally to a cells surface and bearing at least one transmembrane region.
Virus glycoprotein—A protein destined for incorporation into a virus particle that is trafficked through the secretory pathway, normally to a cells surface, and bearing at least one transmembrane region.
Tropism—The ability of a particular virus particle to infect specific cells defines its tropism. Tropism is typically determined by a virus glycoprotein.
Pseudotyping—The incorporation of a non-endogenous surface glycoprotein into a virus particle. Typically, the non-endogenous glycoprotein will provide a new or expanded tropism when compared to the endogenous virus glycoprotein.
Constitutive gene or constitutive expression—a gene that is transcribed continually (as compared to a facultative gene which is only transcribed as needed).

The cell is a mammalian cell. Examples of mammalian cells include those from humans, mice, rats, hamsters, monkeys, rabbits, donkeys, horses, sheep, cows and apes. The cell may be an immortalised cell. Preferably, the cell is a human cell.

In some embodiments, the cell is from an established cell line. Examples of established cell lines include HEK-293, HEK 293T, HEK-293E, HEK-293 FT, HEK-293S, HEK-293SG, HEK-293 FTM, HEK-293SGGD, HEK-293A, MDCK, C127, A549, HeLa, CHO, mouse myeloma, PerC6, 911, and Vero cell lines. Most preferably, the cell line is an unmodified HEK-293, HEK-293A or HEK-293T or a HEK-293 derivative.

The cell or cell line may be one which constitutively expresses Rev. Rev is a transactivating protein that is essential to the regulation of HIV-1 protein expression. A nuclear localization signal is encoded in the rev gene, which allows the Rev protein to be localized to the nucleus, where it is involved in the export of unspliced and incompletely spliced mRNAs. Rev binds to a region in the lentivirus genome called the Rev Response Element which allows the nuclear export of unspliced, full length genomes, which is essential for lentivirus production.

One or more nucleic acid molecules encoding one or more cytotoxic virus polypeptides are introduced into the cell. The cytotoxic virus polypeptides are preferably ones which are each capable of inducing apoptosis of the cell in the absence of the one or more apoptosis inhibitors.

In some embodiments, the cytotoxic virus polypeptide is one whose expression can lead to cell fusion or syncytia formation. In other embodiments, the cytotoxic virus polypeptide is one whose expression can lead to the production of a protease that cleaves proteins within the cell.

In some embodiments, the cytotoxic virus polypeptide is a glycoprotein. Preferably, the cytotoxic virus polypeptide is a membrane protein, more preferably a surface membrane protein. More preferably, the cytotoxic virus polypeptide is a surface membrane glycoprotein. Most preferably, the cytotoxic virus polypeptide is VSV G.

In other embodiments, the cytotoxic virus polypeptide is or comprises a protease. Preferably, the cytotoxic virus polypeptide is Gag-Pol, most preferably from HIV.

In yet other embodiments, the cytotoxic polypeptides are VSV G and Gag-Pol.

In some embodiments, the or each cytotoxic virus polypeptide is independently cytotoxic to the cell at a level of greater than 1, 10 or 100 mg/ml; more preferably greater than 1, 10 or 100 μg/ml; and most preferably greater than 1, 10 or 100 ng/ml or greater than 1, 10 or 100 μg/ml (amounts of polypeptide/ml of cell cytoplasm).

In other embodiments, the or each cytotoxic virus polypeptide is independently cytotoxic to the cell at a level of greater than 1, 10 or 100 mg/cell; more preferably greater than 1, 10 or 100 μg/cell; and most preferably greater than 1, 10 or 100 ng/cell or greater than 1, 10 or 100 μg/cell.

In yet other embodiments, the or each cytotoxic virus polypeptide is independently cytotoxic to the cell at a level of greater than 1, 10 or 100 mmol/cell; more preferably greater than 1, 10 or 100 μmol/cell; and most preferably greater than 1, 10 or 100 nmol/cell or greater than 1, 10 or 100 μmol/cell.

The VSV G polypeptide is a single pass membrane glycoprotein derived from the Vesicular Stomatitis virus. It mediates a broad infectious tropism. As used herein, the term “VSV G polypeptide” refers preferably to a polypeptide having the amino acid sequence given in amino acids 1-511 or 17-511 of SEQ ID NO: 1, or a polypeptide having at least 80%, 85% 90%, 95% or 99% sequence identity thereto (preferably using the BLASTN method of alignment) and which is capable of mediating membrane fusion and/or binding to the low density lipid (LDL) receptor.

The term “Gag-Pol” refers to a retrovirus protein that is proteolytically cleaved to produce a functional reverse transcriptase, integrase, and protease and at least two proteins of structural importance for virus assembly. Preferably, the Gag-Pol sequence is from a lentivirus. It is recognised by those in the art that the Gag-Pol sequence of lentiviruses varies by clade and isolate. All such clades and isolates are encompassed herein. The term “Gag-Pol” refers preferably to a polypeptide having the amino acid sequence given in SEQ ID NO: 2. or a polypeptide having at least 80%, 85% 90%, 95% or 99% sequence identity thereto (preferably using the BLASTN method of alignment). Preferably, the Gag-Pol polypeptide is from HIV.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. (See Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 March; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

VSV G is generally cytotoxic to cells (in the absence of an apoptosis inhibitor). It is capable of inducing cell fusion and the formation of syncytia.

Gag-Pol is generally cytotoxic to cells (in the absence of an apoptosis inhibitor). It is capable of producing a protease that cleaves proteins within the cell and leads to cell death.

The expression of the one or more apoptosis inhibitors mitigates or prevents apoptosis of the cell which would otherwise have been initiated by the cytotoxicity of the cytotoxic polypeptide(s). The one or more apoptosis inhibitors may independently, for example, be a polypeptide or an RNA.

In some embodiments, the apoptosis inhibitor is an inhibitor of the APAF-1 (e.g. AVEN), Caspase 9 (e.g. IAP or XIAP), BAK, BAX or BAD (e.g. BCL2, E1B-19K or BCL-XL) pathway. Preferably, more than one gene is used that inhibits more than one apoptosis pathway or step (e.g. AVEN combined with E1B-19K) to provide improved resistance to apoptosis.

In some embodiments, the one or more of the apoptosis inhibitors is one which inhibits an apoptotic protein whose production is stimulated by loss of cell membrane integrity, by cell-cell fusion or by syncytia formation or one which is stimulated by a protease that cleaves proteins within the cell.

Examples of apoptosis-inhibiting polypeptides include Celovirus GAM1, Adenovirus E4 Orf6, Adenovirus E1B 55K, Adenovirus E1B 19K, Myxomavirus M11L, Cytomegalovirus 1E1, Cytomegalovirus 1E2, Baculovirus p35, Baculovirus IAP-1, Herpesvirus US3, Herpesvirus Saimiri ORF16, Herpes Simplex 2 LAT ORF 1, Human XIAP, African Swine Fever ASFV-5-HL (LMW-5-HL/A179L), Kaposi's Sarcoma virus KSbcl2, Vaccinia virus SPI-2, Cowpoxvirus CrmA, Epstein Barr virus BHRF1, Epstein Barr virus EBNA-5, Epstein Barr virus BZLF-1, Papillomavirus E6, Human Aven, Human BCL2 and Human BCL-XL. Other examples of apoptosis inhibitors include moieties which inhibit the action of the BAX, BAD, BAK or BOK proteins.

In some embodiments, one or more of the apoptosis inhibitors is an RNA, preferably an antisense or shRNA. Other examples of RNA apoptosis inhibitors include Herpesvirus LAT and Adenovirus VA1.

Preferably, one or more of the apoptosis inhibitors is Human Aven and Adenovirus serotype 5 E1B-19K.

Nucleic acid molecules encoding the one or more cytotoxic virus polypeptides and one or more apoptosis inhibitors are introduced into the cell in order to enable expression of the cytotoxic virus polypeptide(s) and the apoptosis inhibitor(s) in the cell. As used herein, the term “introducing” one or more nucleic acid molecules into the cell includes transformation, and any form of electroporation, conjugation, infection, transduction or transfection, inter alia. Processes for such introduction are well known in the art (e.g. Proc. Natl. Acad. Sci. USA. 1995 Aug. 1; 92 (16):7297-301).

In some embodiments of the invention, use may be made of the Cre-Lox system to introduce the one of more nucleic acid molecules encoding the one or more cytotoxic virus polypeptides and/or the one or more apoptosis inhibitors into the cell. In other embodiments of the invention, use may be made of the CRISPR-Cas9 system to introduce the one of more nucleic acid molecules encoding the cytotoxic virus polypeptides and/or the one or more apoptosis inhibitors into the cell.

The nucleic acid molecule(s) may be DNA or RNA. Preferably, it is DNA.

One or more nucleic acid molecules encoding the one or more cytotoxic virus polypeptides and the one or more apoptosis inhibitors are introduced into the cell. For example, a single nucleic acid molecule encoding both the cytotoxic virus polypeptide(s) and the apoptosis inhibitor(s) may be introduced into the cell. In other embodiments, a first nucleic acid molecule encoding the one or more cytotoxic virus polypeptides may be introduced into the cell and a second nucleic acid molecule encoding the one or more apoptosis inhibitors may be introduced into the cell. In yet other embodiments, more than two (e.g. 3, 4, 5 or 6) nucleic acid molecules encoding the one or more cytotoxic virus polypeptides and apoptosis inhibitor(s), or parts thereof, are introduced into the cell. Most preferably, a single nucleic acid molecule encoding both the one or more cytotoxic virus polypeptides and the one or more apoptosis inhibitors is introduced into the cell.

The DNA used in the processes of the invention will be of the B-form and either circular or linear. In the embodiment of the invention using circular DNA, the DNA may be a plasmid, cosmid, expression vector, phagemid, bacterial artificial chromosome (BAC), yeast artificial chromosome (YAC), or any circular DNA molecule capable of replicating in a bacterial host. (The latter forms of DNA will hereinafter be referred to generically as “plasmids and vectors”.) The circular form may also be an enzymatically-recombined form of a plasmid, such as those known in the art as mini-circles.

The DNA may also be replicatable in vitro using virus-derived sequences and paired enzymes, for example phi29 polymerase.

The circular DNA may be linearised prior to introduction into a cell by the polymerase chain reaction or by restriction digestion or similar. It is recognised that mammalian cells linearise DNA once inside the cell. Hence the advantages of delivering linear DNA are marginal, but this allows for control over the specific location of the linearization in the DNA molecule. This prevents the disruption of coding sequences and DNA features of importance in the introduced DNA. Preferably, therefore, the introduced DNA is linear DNA.

In other embodiments, it is preferable to introduce DNA that has been linearised to remove sequences that are responsible for bacterial propagation; and more preferable still, to linearise the DNA in a way that does not affect (e.g. interrupt, destroy, or reduce in anyway) the sequences needed to produce the stable cell line of the invention. These latter sequences include, but are not limited to, promoters, untranslated regions, coding sequences, poly-adenylation sequences, enhancers, insulators and locus control regions (LCR) or ubiquitous chromatin opening element (UCOE).

Preferably, the nucleic acid molecule(s) encoding the one or more cytotoxic virus polypeptides and one or more apoptosis inhibitors are provided on one or more plasmids or vectors.

In some embodiments, the nucleic acid molecules encoding the one or more cytotoxic virus polypeptides and one or more apoptosis inhibitors are provided on the same plasmid or vector.

In other embodiments, the nucleic acid molecules encoding the one or more cytotoxic virus polypeptides and one or more apoptosis inhibitors are provided on different plasmids or vectors. In this case, the plasmids or vectors may be introduced into the cell simultaneously, separately or sequentially.

In yet another embodiment, cells are produced which express the one or more apoptosis inhibitors; those cells are subsequently modified further to introduce the nucleic acid molecule encoding the one or more cytotoxic virus polypeptides.

The nucleic acid molecule(s) encoding the one or more cytotoxic virus polypeptides and the one or more apoptosis inhibitors may independently be operably-associated with one more regulatory sequences, e.g. promoters, enhancers and terminators. The nucleic acid molecule(s) encoding the one or more cytotoxic virus polypeptides and one or more apoptosis inhibitors may independently be in the form of an expression cassette.

The cell expresses the one or more cytotoxic virus polypeptides constitutively.

Constitutive expression of the one or more cytotoxic virus polypeptides may be driven by one or more constitutive (non-inducible) promoters.

Examples of suitable promoters the CMV, SV40, PGK (human or mouse), HSV TK, SFFV, Ubiquitin, Elongation Factor Alpha, CHEF-1, FerH, Grp78, RSV, Adenovirus EIA, CAG or CMV-Beta-Globin promoter, or a promoter derived therefrom. Preferably, the promoter is the Cytomegalovirus immediate early (CMV) promoter, or a promoter which is derived therefrom, or a promoter of equal or increased strength compared to the CMV promoter.

The cell expresses the one or more apoptosis inhibitors constitutively. Constitutive expression of the one or more apoptosis inhibitors may be driven by one or more constitutive promoters.

Preferably, each apoptosis inhibitor promoter is one which is selected such that it provides the optimum expression level of the associated nucleic acid encoding the apoptosis inhibitor.

Examples of apoptosis inhibitor promoters include the CMV, SV40, PGK (human or mouse), HSV TK, SFFV, Ubiquitin, Elongation Factor Alpha, CHEF-1, FerH, Grp78, RSV, Adenovirus EIA, CAG or CMV-Beta-Globin promoters. Preferably, the apoptosis inhibitor promoters are selected from the group consisting of RSV, CMV, SV40, PGK and ubiquitin promoters. Particularly preferred promoters are CMV and SV40.

In embodiments of the invention wherein more than one apoptosis inhibitor is used, each apoptosis inhibitor is preferably driven independently by a different promoter; and each promoter is preferably of a different type (e.g. CMV, SV40, etc.).

The ability of a number of apoptosis inhibitors to work effectively when driven from promoters of varying strengths has been shown herein to range from low to high. It is shown that some apoptosis-inhibiting genes can vary in anti-apoptotic activity by as much as 0.5-log depending on which promoter is used to drive expression. Promoters including the RSV, Ubiquitin, CMV, SV40 and PGK promoters were used and these were found to vary by as much as 15-fold in terms of relative transcription and subsequent protein expression, with some apoptosis inhibiting genes demonstrating optimal activity at low concentrations and others performing optimally at high concentrations. It is preferable, therefore, that each nucleic acid encoding an apoptosis inhibitor is expressed under the control of a promoter that provides the cell with optimal apoptosis inhibition.

In some preferred embodiments, the processes of the invention comprise the use of the following promoter-apoptosis inhibitor combinations:

### RSV—Human Aven

RSV—Adenovirus serotype 5 E1B-19K

### CMV—Human Aven

CMV—Adenovirus serotype 5 E1B-19K

### SV40—Human Aven

SV40—Adenovirus serotype 5 E1B-19K

### PGK—Human Aven

PGK—Adenovirus serotype 5 E1B-19K

### Ubiquitin—Human Aven

Ubiquitin—Adenovirus serotype 5 E1B-19K.

Preferably, the cell expresses both human Aven and Adenovirus serotype 5 E1B-19K, wherein the expression of human Aven and Adenovirus serotype 5 E1B-19K is driven by different promoters selected from RSV, CMV, SV40, PGK, GRP78, EF1-Alpha, SFFV, CHEF-1, Adenovirus EIA, Chicken Beta Actin, CAG, CMV-Beta-Globin, and ubiquitin promoters.

In yet other embodiments, the apoptosis inhibitor is not Aven or Adenovirus serotype 5 E1B-19K.

In these embodiments, particularly preferred apoptosis inhibitors include use of one or more apoptosis inhibitors selected from the group consisting of KSbcl2, BHRF1, XIAP, BCL-XL, ASFV-5-HL and Vaccinia virus SPI-2.

In some such preferred embodiments, the processes of the invention comprise the use of the following promoter-apoptosis inhibitor combinations:

### CMV—BHRF1,

### CMV—or SV40—KSbcl2,

### CMV—Vaccinia SP2,

### CMV—ASFV-5-HL,

### SV40—XIAP,

### SV40—M11L, or

SV40—SV40 Large T antigen,
either singly or in combination with other apoptosis inhibitors.

In some embodiments, it is preferable to combine pairs of apoptosis inhibitor genes under the control of relatively strong (e.g. CMV) and relatively weak (e.g. SV40) promoters to give comparatively high and low levels of expression, respectively, of the two apoptosis inhibitor genes. Preferred combinations in this regard include highKSbc12+lowXIAP; highKSbc12+lowM11L; and high BHRF1+lowKSbc12. Other preferred combinations include high BHRF1+lowXIAP; highASFV5HL+lowXIAP; and highKSbcl2+lowlAP1. Preferably, the high level of expression is provided by the CMV promoter. Preferably, the low level of expression is provided by the SV40 promoter.

Preferably, the nucleic acid molecule(s) encoding the one or more cytotoxic virus polypeptides and the one or more apoptosis inhibitor(s) become stably integrated into the host cell genome.

Preferably, the nucleic acid molecule(s) encoding the one or more cytotoxic virus polypeptides and the one or more apoptosis inhibitor(s) additionally comprise a selection gene.

The production of stable cell lines in mammalian culture typically requires a method of selection to promote the growth of cells containing any exogenously added DNA.

To this end, a range of genes are known that provide resistance to specific compounds when the DNA encoding them is inserted into a mammalian cell genome.

Preferably, the selection gene is puromycin N-acetyl-transferase (Puro), hygromycin phosphotransferase (Hygro), blasticidin s deaminase (Blast), Neomycin phosphotransferase (Neo), glutathione S-transferase (GS), zeocin resistance gene (Sh ble), or dihydrofolate reductase (DHFR). Each of these genes provides resistance to a small molecule known to be toxic to mammalian cells, or in the case of GS provides a method for cells to generate glutathione in the absence of glutathione in the growth media.

In a preferred embodiment of the invention, the resistance gene is Puro. This gene is particularly effective because many of the cell lines used in common tissue culture are not resistant; this cannot be said for Neo where many, particularly HEK 293 derivatives, are already Neo resistant due to previous genetic manipulations by researchers (e.g. HEK 293T cells). Puro selection also has the advantage of being toxic over a short time window (<72 hours), and hence it allows variables to be tested rapidly and cells that do not harbour the exogenous DNA to be inserted into the genome are rapidly removed from the culture systems. This cannot be said of some other selection methods such as Hygro, where toxicity is much slower onset.

In one embodiment of the invention, the cells are selected using a puromycin concentration of 0.5-10 μg/ml of puromycin in the culture media. In a more preferred embodiment of the invention, the amount of puromycin used in the culture media is between 2-6 μg/ml. The most preferred concentration is 3-5 μg of puromycin per ml of culture media.

The development of stable cell lines using selection genes (e.g. Puro) requires that the resistance gene must be expressed in the cells. This can be achieved through a variety of methods including, but not limited to, internal ribosome entry sites (IRES), 2A cleavage systems, alternative splicing, and dedicated promoters.

In a particularly preferred embodiment of the invention, the expression of the one or more cytotoxic virus polypeptides is linked to the expression of the selection gene and they are transcribed in the same primary transcript.

In a particularly preferred embodiment of the invention, the selection gene is inserted after (3′) an IRES downstream (3′) of the last stop codon of the nucleic acid encoding one or more cytotoxic virus polypeptides. This provides a configuration where a promoter initiating transcription is upstream (5′) to the coding sequences of the one or more cytotoxic virus polypeptide gene(s) which is then followed (3′) by an IRES which is then followed (3′) by the coding region for selection/resistance gene allowing for cell selection (preferably using Puro). In this configuration, both the one or more cytotoxic virus polypeptide and selectable marker are encoded by the same mRNA, but due to the relatively low efficiency of IRES-mediated translation the one or more cytotoxic virus polypeptides will be translated in greater abundance than the selectable marker. To maintain a selectable phenotype, this will ensure that expression of the one or more cytotoxic virus polypeptides continues at a high level and cannot be silenced. A stable cell line, engineered according to the invention in this way and selected for using a marker, would therefore necessarily combine high levels of expression of the one or more cytotoxic virus polypeptides alongside expression of one or more anti-apoptosis factors to allow cell survival. In that way, the selectable marker selects cells that express high levels of the one or more cytotoxic virus polypeptides and thereby also expression of apoptosis-resistance.

Alternative methods include enabling alternative splicing such that from within the same primary transcript two or more alternatively spliced mRNA molecules are produced at a given ratio that enable the one or more cytotoxic virus polypeptides to be produced from the primary transcript as a resistance gene (e.g. Puro).

In another embodiment, a coding sequence may be constructed that allows the coding sequences of the one or more cytotoxic virus polypeptide genes to be linked in-frame with the coding sequence of a resistance or selection gene (e.g. Puro) using self-cleaving protein sequences (such as that derived from the 2A sequence of Foot and Mouth Disease (FMDV)). In producing this coding sequence, it will be possible to express two or more genes from the same coding region and gene region. However, in this embodiment, the polypeptides encoded by the one or more cytotoxic virus polypeptide genes and the resistance or selection gene would be produced at equimolar concentrations. This is not an ideal configuration because the invention preferably aims to maximise the expression of the one or more cytotoxic virus polypeptides and, in order to enable this, it is desirable to have lower amounts of the resistance protein compared to the one or more cytotoxic virus polypeptides.

In other preferred embodiments, the nucleic acid encoding the one or more cytotoxic virus polypeptide genes and resistance/selection gene have independent promoters.

For example, in the cell line HEK-293, compared to the expression level of the CMV promoter (a preferred promoter to drive one of the one or more cytotoxic virus polypeptide genes), the relative expression levels of the Herpes Simplex virus Thymidine Kinase (HSV TK), Human Ubiquitin C (Ubc) and the Simian virus 40 (SV40) promoters are approximately 19-, 18- and 12-fold lower, respectively. The latter promoters are therefore preferred promoters to drive the expression of a resistance/selection gene because they result in considerably lower expression levels, forcing the cell to express high levels of the one or more cytotoxic virus polypeptides compared to the lower levels of the selection gene.

However, the use of individual promoters has a disadvantage because when the DNA encoding these gene regions is inserted into the mammalian cell genome: it may be the case that only the resistance gene becomes inserted into the genome. It is also possible that the nucleic acids encoding the one or more cytotoxic virus polypeptides becomes silenced by epigenetic modification of the DNA, whilst the resistance/selection gene remains active. This can occur because the genes are not part of the same primary transcript molecule, where it is not possible to express one without the other.

Therefore, it is particularly preferred to use an IRES to drive the expression the resistance/selection gene and the CMV promoter (or a promoter with equal or higher strength derived from the CMV promoter) to drive expression of the nucleic acid encoding the one or more cytotoxic virus polypeptide genes, with both the resistance/selection marker and the one or more cytotoxic virus polypeptides encoded by the same mRNA. The reasons for the preferred use of an IRES is because they typically demonstrate at least 20-fold lower protein expression compared to the upstream (5′) gene and yet they are genetically linked, being on the same mRNA molecule but translated via an alternative cap-independent method. This means that when cells are resistant to a given toxic compound because of the integration of a resistance/selection gene they must (or are more likely) to also be expressing the one or more cytotoxic virus polypeptides. In this way expression of the one or more cytotoxic virus polypeptides gene cannot be silenced while expression of the selectable marker persists, and it will continue at a high level. Theoretically it is possible that the IRES and resistance/selection gene may integrate into the mammalian cell genome independent of the one or more cytotoxic virus polypeptides gene. However, the nature of IRESs means that in order to have protein expression, the nucleic acid must insert into an existing gene's exon, which is highly unlikely. If using splicing systems or independent promoters, it is considerably more likely that aberrant expression could be observed by integration into the host genome at either transcriptionally-active regions, or regions that support active transcription, respectively.

In Step (ii), the cells are cultured under conditions such that the one or more cytotoxic virus polypeptides and the one or more apoptosis inhibitors are all expressed.

In the absence of the apoptosis inhibitor(s), the expression of the cytotoxic virus polypeptides in the cell would ordinarily result in cytotoxic events (such as cell-cell fusion and the production of syncytia) leading to the apoptosis of the cell. The co-expression of the one or more apoptosis inhibitors prevents this apoptosis.

The expression of the one or more cytotoxic virus polypeptides in the cell may be determined by a number of different methods.

Such methods include using antibodies to the VSV G polypeptide in immunohisto-chemical (IHC) processes and FACS.

In particular, the presence of the VSV G polypeptide in the cell may be demonstrated by the presence of cell-to-cell fusions or the formation of multi-cellular syncytia. VSV G expression may also be shown by staining the cells with an antibody specific to the VSV G glycoprotein.

Gag-Pol may be detected by ELISA or western blot using antibodies raised specifically against these proteins. To specifically measure Gag protein, a p24 assay exists which is both highly sensitive and re-producible and allows the rapid detection and quantitation of Gag protein in cells, supernatant and virus preparations.

In other embodiments, the invention provides a cell line wherein the cells of the cell line constitutively express one or more cytotoxic virus polypeptides and one or more apoptosis inhibitors, and wherein the expression of the apoptosis inhibitor(s) prevents apoptosis of the cells of the cell line. Preferably, the cells of the cell line are capable of expressing one or more of the cytotoxic polypeptides at a level which is high enough for the pseudotyping of retrovirus particles.

Preferably, cells of a cell line constitutively expressing VSV G and one or more apoptosis inhibitors are able to support virus production of at least 1×10⁵ virus particles/ml after 48 hours post transfection of the virus genome.

Preferably, cells of a cell line constitutively expressing Gag-Pol and one or more apoptosis inhibitors are able to support virus production of at least 1×10⁵ virus particles/ml after 48 hours post transfection of the virus genome.

Preferably, the cell line is one which is capable of being passaged at least 5×, more preferably at least 10× and most preferably at least 15×.

In yet other embodiments, the invention provides a nucleic acid molecule encoding one or more cytotoxic virus polypeptides and one or more apoptosis inhibitors.

In yet other embodiments, the invention provides a kit comprising:
(i) a nucleic acid molecule encoding one or more cytotoxic virus polypeptides, and
(ii) a nucleic acid molecule encoding one or more apoptosis inhibitors,
   for use in simultaneously, sequentially or separately introducing the nucleic acid molecules into a mammalian cell, in order to produce cells which constitutively express the one or more cytotoxic virus polypeptides and the one or more apoptosis inhibitors, and wherein the expression of the apoptosis inhibitor(s) prevents apoptosis of the cells.

A key use of the cells of the invention is in the production of retroviral vectors.

In a further embodiment, therefore, there is provided a process for producing retroviruses, the process comprising the steps:
(a) introducing one or more nucleic acids encoding a retrovirus into a cell or a cell of a cell line of the invention;
(b) introducing one or more helper plasmids encoding one or more of Rev, Gag-Pol or Tat polypeptides into the cell;
(c) culturing the cell under conditions such that retroviruses are assembled and secreted by the cell; and
(d) harvesting packaged retrovirus from the supernatant.

Preferably, the retrovirus is a Lentivirus.

Lentiviruses are a subset of the retroviridae family that are increasingly used for transgene delivery and protein expression, particularly in progenitor cell populations such as haematopoietic stem cells and T cells. Unlike most retroviruses, lentiviruses are able to deliver their genome, or modified forms thereof, independent of the cell cycle, and often achieve higher efficiency of cellular infection in a shorter time frame. This makes them a much more effective viral vector for both research and clinical use.

The lentivirus family consists of 10 viruses at present. These species are divided into five groups including: Bovine lentivirus group (Bovine immunodeficiency virus and Jembrana disease virus), Equine lentivirus group (Equine infectious anemia virus, Feline lentivirus group, Feline immunodeficiency virus, Puma lentivirus), Ovine/caprine lentivirus group (Caprine arthritis encephalitis virus, Visna/maedi virus), Primate lentivirus group, (Human immunodeficiency virus 1, Human immunodeficiency virus 2, Simian immunodeficiency virus).

In a preferred embodiment, the process of the invention is used to generate a cell line for the production of Human immunodeficiency virus 1, Simian immunodeficiency virus or Equine infectious anemia virus viral vectors. In a more preferable embodiment, the process is used to produce cell lines for the production of Human immunodeficiency virus 1 or Equine infectious anemia virus viral vectors.

The one or more nucleic acids encoding a retrovirus may also encode a desired polypeptide.

The nucleic acids encoding the retrovirus may comprise a self-inactivating retroviral genome.

Preferably, one of the helper plasmids encodes VSV G. In other embodiments, preferably one of the helper plasmids encodes Gag-Pol. Preferably, one of the helper plasmids encodes Rev. Preferably, one of helper plasmids encodes Tat.

In some embodiments, one helper plasmid is used: this encodes Gag-Pol, Tat and Rev. In some other embodiments, one helper plasmid is used: this encodes VSV G, Tat and Rev.

In other embodiments, two helper plasmids are used: the first encodes Gag-Pol; the second encodes Rev. In this embodiment, the retroviral genome preferably has a 5′LTR which includes a promoter (thus obviating the need for the Tat protein).

In yet other embodiments, two helper plasmids are used: the first encodes VSV G; the second encodes Rev. In this embodiment, the retroviral genome preferably has a 5′LTR which includes a promoter (thus obviating the need for the Tat protein).

In yet another embodiment one helper plasmid used: this encodes the retrovirus genome and Gag-Pol.

Methods for culturing of the cells and harvesting the desired polypeptides are well known in the art.

Preferably, the harvested retroviruses are subsequently purified.

The invention also provides a process for producing a recombinant polypeptide, the process comprising the steps:
(a) producing retroviruses by a process of the invention, wherein the retroviral genomes comprise a nucleic acid molecule encoding a desired recombinant polypeptide;
(b) introducing the retroviruses into a host cell;
(c) culturing the host cell under conditions such that the desired recombinant polypeptide is produced; and
(d) harvesting the desired polypeptide.

Examples of suitable host cells include BHK and CHO cells.

In some embodiments, the desired recombinant polypeptide is not a non-cytotoxic polypeptide. In other embodiments, the desired recombinant polypeptide is not a blood-clotting protein. In other embodiments, the desired recombinant polypeptide is not Factor VIII.

The disclosure of each reference set forth herein is incorporated herein by reference in its entirety.

### BRIEF DESCRIPTION OF THE FIGURES

FIG. 1: Apoptotic efficiency of various gene/promoter combinations.

FIG. 2: Apoptotic efficiency of various gene/promoter combinations.

FIG. 3: Expression of VSV G in cell lines using antibodies against VSV G glycoprotein.

FIG. 4: Lentivirus production in transfected cell lines.

FIG. 4A: Cells infected with virus produced using standard four-plasmid lentivirus recovery.

FIG. 4B: Virus from stable cell line 1: produced by integration of genes encoding VSV and two genes known to inhibit apoptosis selected via puromycin resistance.

FIG. 4C: Virus from stable cell line 1: produced by integration of genes encoding VSV G and two genes known to inhibit apoptosis selected via puromycin resistance.

FIG. 5: Flow cytometry of cells infected with GFP expressing virus.

FIG. 5A: Cells infected with virus produced using standard four plasmid lentivirus recovery.

FIG. 5B: Virus from stable cell line 1, produced by integration of genes encoding VSV and two genes known to inhibit apoptosis selected via puromycin resistance.

FIG. 5C: Virus from stable cell line 1, produced by integration of genes encoding VSV G and two genes known to inhibit apoptosis selected via puromycin resistance.

FIG. 6: Stable cell lines expressing VSV G seeded at high density or confluence leads to the formation of large multi-nucleated syncytia.

FIG. 7: A plasmid schematic of a DNA molecule that could be used in the process of the invention by introduction into a mammalian cell. The diagram shows a DNA molecule encoding the VSV G glycoprotein and the apoptosis inhibitors human AVEN and Adenovirus E1B-19K.

FIG. 8: A plasmid schematic of a DNA molecule that could be used in the process of the invention by introduction into a mammalian cell. The diagram shows a DNA molecule encoding the VSV G glycoprotein and containing a Puromycin resistance gene downstream of an internal ribosome entry site (EMCV IRES) in addition to the apoptosis inhibitors human AVEN and Adenovirus E1B-19K.

FIG. 9: The effect of a range of genes (apoptosis inhibitors) on protein expression (luciferase) where each gene is under the control of the strong CMV promoter.

FIG. 10: The effect of a range of genes (apoptosis inhibitors) on protein expression (luciferase) where each gene is under the control of the weak SV40 promoter. Luciferase is driven by the CMV promoter.

FIG. 11: The effect of combining pairs of apoptosis inhibitor genes under the control of CMV and SV40 promoters, to give comparatively high and low levels of expression, respectively. Luciferase expression is driven by the CMV promoter.

FIG. 12: Human IgG antibody expression in suspension HEK-293 cells using a specific apoptosis inhibitor combination, namely KSbcl2 and BHRF1 both under regulatory control of the CMV promoter. Lanes 1: ladder. Lanes 2&3: competitor's plasmid at 12 μg. Lanes 3&4: competitor's plasmid at 24 μg. Lanes 15&16: plasmid containing a specific apoptosis inhibitor combination at 12 μg. Lanes 17&18: plasmid containing a specific apoptosis inhibitor combination at 24 μg. Apoptosis inhibitors allow the usage of higher DNA quantities without seeing the reduction in protein expression seen in Lanes 4&5.

FIG. 13: Yellow fever NS1 secreted protein expression measured via ELISA after 3 days from suspension HEK-293 cells. Sample A contains a standard CMV expression plasmid. Sample K contains a plasmid encoding KSbcl2 apoptosis inhibitor under regulatory control of the CMV promoter, shown here to significantly increase protein expression.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Materials and Methods

### DNA Engineering

All DNA constructs were synthesised by de novo synthesis where required. This process involved thermodynamically-balanced inside out oligo assembly to allow full length constructs to be produced from smaller oligos nucleotides (typically 4-50 nt in length). For the joining of DNA molecules, a combination of type II restriction endonuclease cloning and assembly PCR were used. The accuracy of each DNA construct was verified by restriction digestion and agarose electrophoresis and/or DNA sequencing using the Sanger method. All protocols are known to those in the art (e.g. Molecular Cloning: A Laboratory Manual (Fourth Edition), Michael R. Green, Joseph Sambrook, Cold Spring Harbor Laboratory Press).

### Protocol for Creating Cell Lines

In order to establish a stable cell line expressing VSV G, HEK 293 cells were seeded in Dulbecco's Modified Eagle (DMEM) media (10% FCS, 1% penicillin/streptomycin) into T25 flasks (10 cm or 6 cm dishes may also be used) 24 hours prior to transfection so as to be at 80% confluent at time of transfection.

Cells were transfected using the PEI method. Briefly, the transfection mixture consisted of 15 μg plasmid DNA in a 1:3 ratio with Branched PEI (25 KDa) respectively, added to two vials of 150p1 of DMEM (2% foetal calf serum (FCS)) Optimem media. Other media may be used, and preferably the media used would be Optimem to complex the DNA and would be free of both FCS and Penicillin and/or Streptomycin. The media/DNA mix and the media/PEI mix were combined and was incubated for 20 minutes at room temperature to allow complex formation. At the time of transfection, the pre-existing media in which the cells were seeded was removed by aspiration and changed for fresh DMEM media containing 10% FCS. Transfection mixtures were added drop-wise into the flasks and gently swirled to evenly distribute the transfection complexes in the media.

24 hours post transfection, the media and transfection mixtures in each flask was removed by aspiration and replaced with DMEM (10% FCS, 1% penicillin/streptomycin) media containing puromycin which was added to the flasks at varying concentrations to determine the optimal antibiotic concentration required. For each DNA plasmid being tested, one flask was maintained at each of the following concentrations: 0 μg/ml, 0.5 μg/ml, 1.5 μg/ml, 3 μg/ml, 5 μg/ml, 7.5 μg/ml and 10 μg/ml puromycin per ml of growth media.

Over the next 4 weeks, media in the flasks were changed every 3-4 days, maintaining the same concentrations of puromycin relevant to each flask, and the flasks were continuously evaluated for cell death and formation of cell foci/colonies. Formation of foci from single surviving cells was clearly observed in the flasks maintained at either 3 μg/ml and 5 μg/ml puromycin in flasks transfected with plasmids containing VSV G and the apoptosis inhibitors. After 4 weeks, two alternative approaches were taken. The contents of each of flask were either:
1. Gently trypsinized and passaged into larger T75 flasks, to make mixed population cell lines, maintaining the same puromycin concentration for each line. This was then followed by single cell isolation by limited dilution or FACS to identify single monogenic cell lines; or
2. Single colonies were encircled by a polycarbonate ring fixed temporarily to the flasks surface with sterile grease, followed by trypsinisation and colony isolation from within the ring and transfer to a 6-well plate containing the same media containing puromycin. The latter is the more preferable method of clonal cell line isolation.

After cells reached sufficient confluence, they were passaged with a 5-fold dilution; the remaining cells were analysed by flow cytometry. The cell lines were maintained from this point onwards at the same concentration of puromycin as originally selected in. Cell banks were created and stored at −170° C. using the cells remaining from each passage. In some instances, it may be possible to increase the VSV G expression by increasing the puromycin concentration in 1-2 μg/ml increments, selecting for only the cells expressing the highest quantity of VSV G.

### Protocol for Microscopy of Syncytia and GFP

Cell lines selected as described previously were seeded in 6 cm dishes so as to be 100% confluent after 24 hrs. The cell monolayer was observed for the formation of syncytia on a daily basis. After 5 days, cells were imaged using a Zeiss Axiovert Inverted microscope and imaged using a NIKON Coolpix camera with microscope adaptor. Where GFP images were required, samples were excited using ultraviolet excitation.

### Protocol for FACS Analysis

A confluent T25 flask of cells from each cell line selected to express VSV G, as well as a flask of wild-type unmodified HEK 293 cells, were trypsinized and 5 ml of cell suspension were collected in 15 ml falcon tubes. These tubes were shaken at 37° C. for 1 hr to allow recovery of the VSV G glycoprotein on the cell surface after the trypsinisation process.

Each tube containing cells were then centrifuged at 1500 RPM for 5 minutes. Once pelleted, supernatants were removed by gentle aspiration and tubes were placed on ice. Cell pellets were re-suspended in PBS; 500p1 for the VSV G expressing cells and 750p1 for the HEK 293 cells. 250p1 amounts of the re-suspended cells were then aliquoted into 1.5 ml polypropylene tubes.

In order to stain cells to measure VSV G protein on the cell surface, solutions of primary antibody (anti-VSVG), secondary antibody (anti-mouse, FITC labelled) and isotype control (IgG) antibody were prepared at 4 μg/ml in MACS buffer.

The cells in 1.5 ml polypropylene tubes were spun down at 5000 RPM in a benchtop centrifuge, the supernatant was removed, and the cell pellets were resuspended either in 200p1 MACS buffer+primary Ab or 200p1 MACS+isotype control Ab. Additionally, one tube of unmodified HEK 293 cells was suspended in 200p1 MACS alone, as an unstained cell control. The tubes were incubated at 12° C., shaking at 300 RPM, for 30 minutes.

After incubation, the samples were pelleted at 5000 RPM and washed twice in 250p1 PBS, pelleting between washes as described before. Samples were retained on ice as much as possible. Both the test and the control samples (apart from the unstained unmodified HEK 293 negative control cells) were then resuspended in MACS+ secondary Ab and incubated again at 12° C., shaking at 300 RPM, for 30 minutes. After the second incubation, the cells were pelleted as before and washed twice in PBS, and resuspended in a final volume of 250p1 PBS. Samples were analysed on a BD FACSCalibur using an Argon 488 Laser, gated for positivity against unstained HEK 293 cells.

In most experiments, stained cells were ready immediately after the staining protocol. However, when this was not possible (e.g. time periods over 4 hours between staining and analysis), cells were fixed by incubating in PBS 2% paraformaldehyde (PFA) for 15 minutes on ice, with gentle swirling to mix. The cells were then spun down and washed twice in PBS as before, resuspended in PBS, and stored at 4° C. Results from both methods consistently demonstrated high levels of VSV G on the surface of analysed VSV G cell lines.

### Example 2: Apoptosis Inhibiting Genes

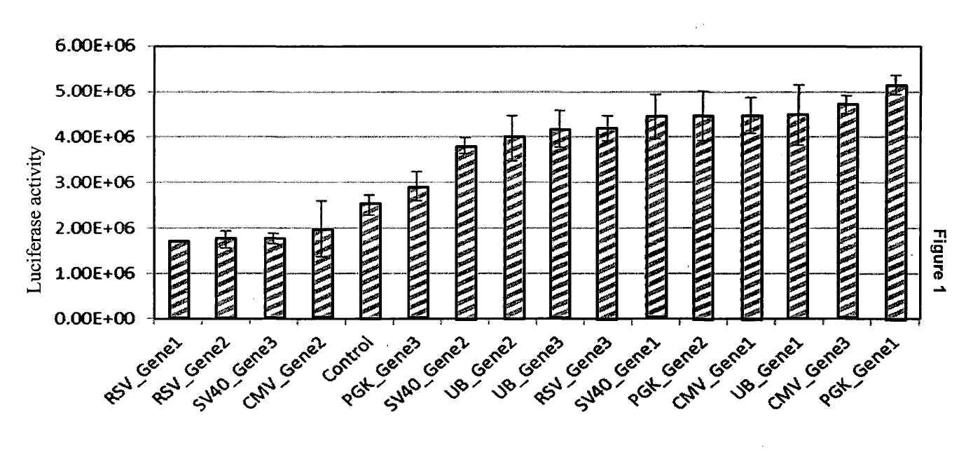

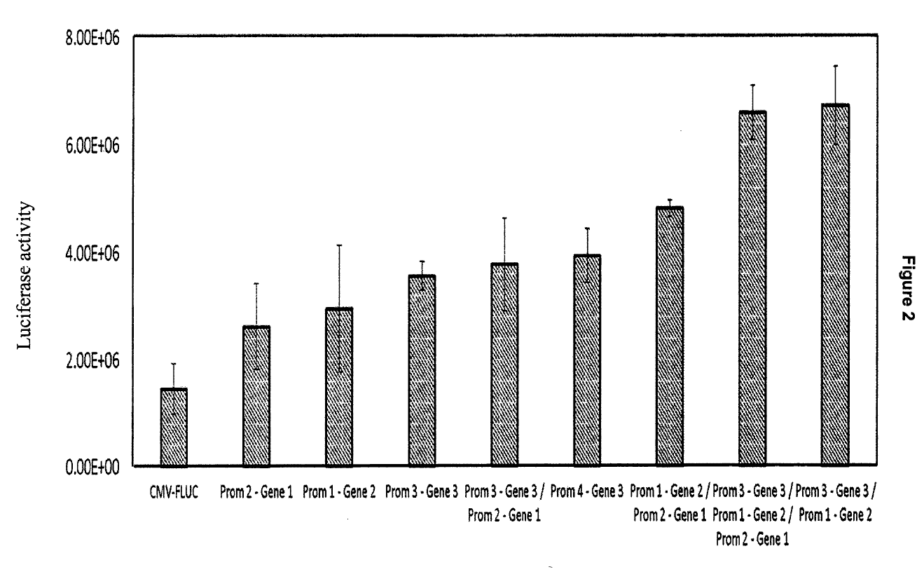

Gene expression was measured as a function of cell survival from apoptosis. Different genes inhibiting apoptosis were found to work with different efficiencies depending on their level of expression. For example, FIG. 1 shows that Gene 3 shows much improved activity under the promoter showing the highest expression level (CMV), whereas the weakest promoter (SV40) produces only a modest protection against apoptosis. However, Gene 2 shows the converse of this with the promoters showing the lowest expression level (SV40 and PGK) yielding the greatest protection against apoptosis. (In FIGS. 1-2, Gene 1 is Bcl-XL, Gene 2 is E1B-19K and Gene 3 is AVEN.)

FIG. 2 demonstrates that, when some inhibitors of apoptosis are combined, they can demonstrate improved resistance to apoptosis. In this assay, there is a strong trend towards cells expressing Gene 3 (AVEN) and Gene 2 (E1B-19K) showing improved resistance to apoptosis in comparison to either gene alone.

### Example 3: Expression of VSV G in Cell Lines

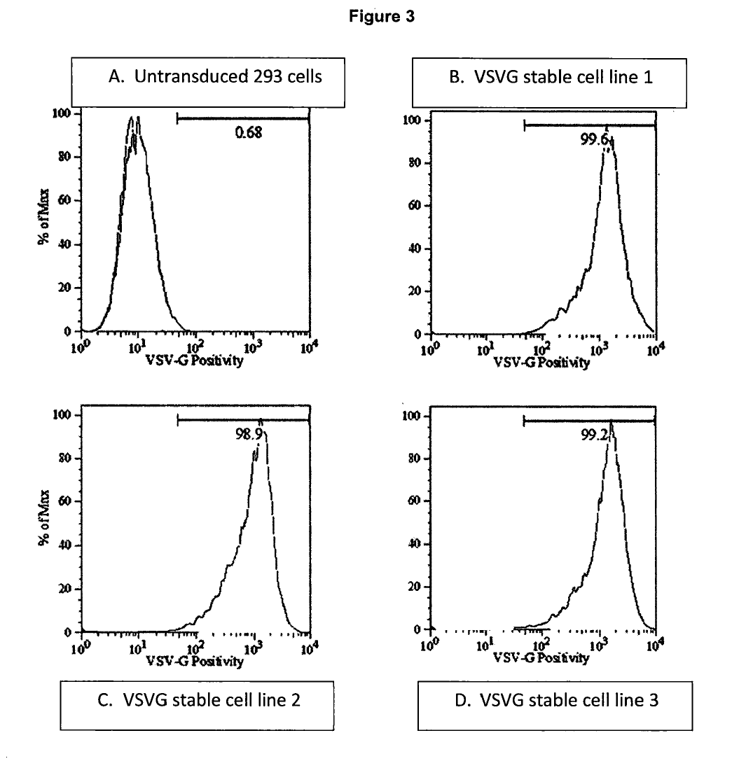

FIG. 3 shows 4 individual cell lines (HEK 293 cells and stable cell lines expressing VSV G) which were stained using an antibody against the VSV G glycoprotein. In all cell lines produced to express VSV G, >98% of cells were highly positive for VSV G. HEK 293 cells showed no staining and an isotype control confirmed that the signal was specific to VSV G.

### Example 4: Expression of Recombinant Lentiviruses in Transfected Cell Lines

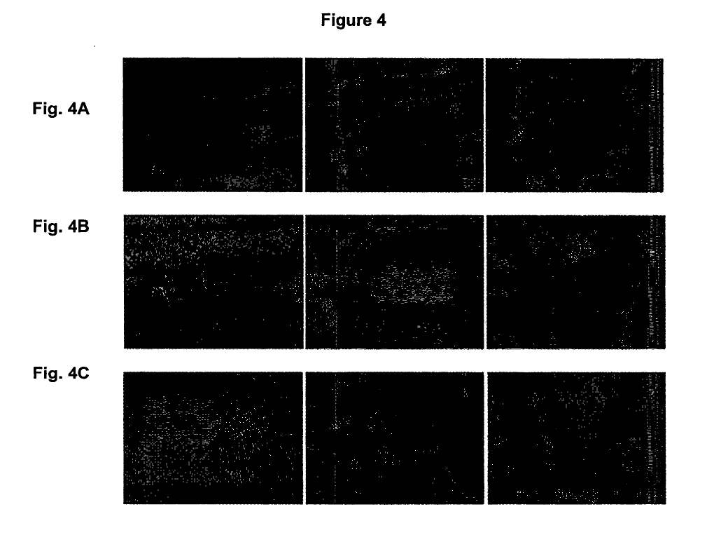

HEK 293 cells were infected with a lentivirus expressing GFP that had been produced by either standard 4 plasmid transfection into HEK 293 cells (includes the VSV G plasmid) and compared to lentivirus produced in cell lines modified to constitutively express VSV G via 3 plasmid transfection (excludes the VSV G plasmid). FIG. 4 shows that, in all cases, more virus was found to be produced from cell lines which were constitutively expressing VSV G.

### Example 5: Flow Cytometry of Cells Infected with GFP Expressing Virus

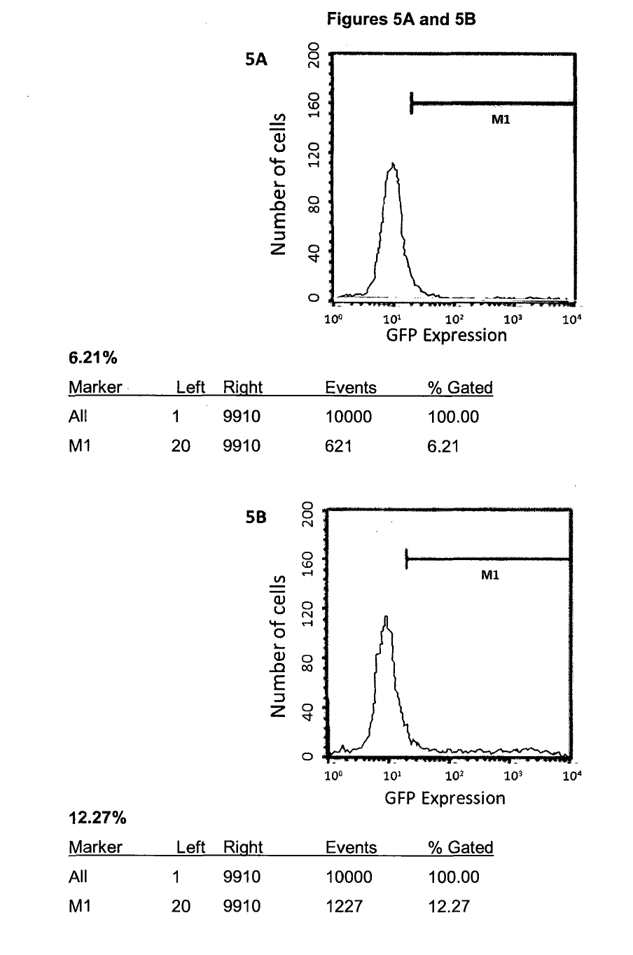

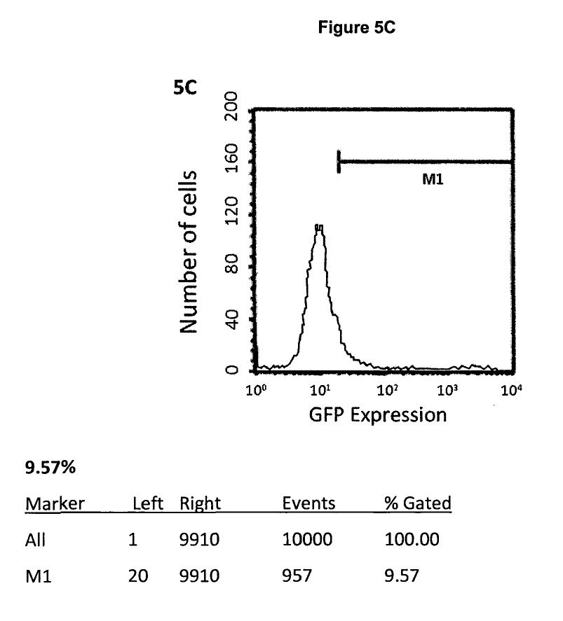

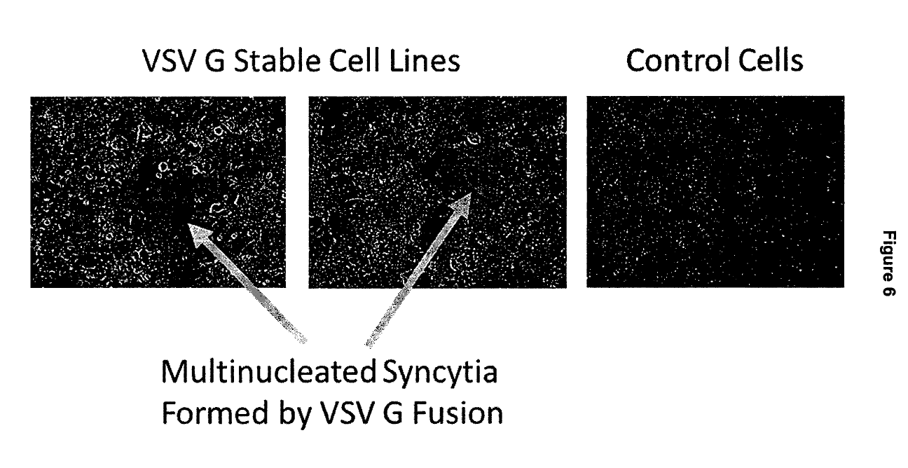

Cells infected with GFP-expressing virus produced by either standard 4-plasmid transfection into HEK 293 cells (includes the VSV G plasmid) or transfection of stable cell lines modified to express VSV G using a 3-plasmid transfection (excludes the VSV G plasmid) were analysed by flow cytometry. The results (FIG. 5) show that significantly more virus was produced from the stable VSV G cell lines in comparison to the industry-standard (i.e. 4-plasmid) method.

### Example 6: Preliminary Screening for Lentivirus Production

VSV G expressing cell lines and wild-type HEK 293 cells were seeded at equal numbers in DMEM media (10% FCS, 1% penicillin/streptomycin) into three 6-well plates 24 hours prior to transfection so as to be at 80% confluence at the time of transfection 24 hours later. On each 6 well plate, 3 wells were seeded with wild-type HEK 293 cells, and each of the remaining 3 wells was seeded with cells from one of three selected VSV G expressing cell lines.

At time of transfection, DNA/PEI complexes were made up as follows:

For the VSV G expressing cells transfections, the following amounts of DNA/well were added to 395p1 of optimum per well in a master solution:
0.79 μg Lentivirus GFP vector
0.79 μg Rev plasmid
1.9 μg Gag/pot plasmid

An amount of branched PEI equivalent to 3× the total weight of DNA was added to another 395p1 of optimum per well in a master solution for all wells.

Both the PEI and DNA solutions were filtered through a 0.2 micrometer sterile filter, and then the PEI solution was added dropwise to the DNA solution.

A solution which was identical in composition but also including 0.2 micrograms of VSV G plasmid was used for the transfection of the standard HEK 293 cell lines to allow comparison to standard lentivirus production systems.

Additional cells of HEK 293 cells were also seeded to act as negative controls and others seeded to generate a GFP positive control. For this control, a transfection complex identical to that described above using 7.4 μg of a CMV-GFP plasmid vector only was used.

All transfection complexes were incubated for 20 minutes at room temperature in which time the seeded cells were washed with optimum medium, then 835p1 of each transfection complex was added dropwise to the respective wells. Cells were left to incubate at 37° C., 5% CO₂ overnight and in the morning approximately 16-18 hours after transfection, the media was changed for DMEM (10% FCS, 1% penicillin/streptomycin). Supernatant from each well was then harvested at 48 hours post transfection and replaced with fresh media and harvested again at 72 hours after transfection. Supernatants were stored at −20° C.

In order to analyse the level of virus produced from each cell line, unmodified HEK 293 cells were seeded in DMEM (10% FCS, 1% penicillin/streptomycin) to a density of 90% in a 48 well plate 24 hours prior to infection. Two dilutions of harvested supernatant from each time point were used to infect the cells in triplicate wells. Dilutions were 2/5 and 4/25 into DMEM (10% FCS, 1% penicillin/streptomycin). Cells were infected by removing the overnight media, and adding 500p1 of diluted supernatant.

Cells were incubated at 37° C., 5% CO₂ for 48 hours. After 48 hours post infection, the cells were tested for GFP expression by flow cytometry. Samples were analysed on a BD FACSCalibur using an Argon 488 Laser, gated for positivity against unstained HEK 293 cells. The level of GFP positive cells was then used to calculate virus titre.

### Example 7: Lentivirus Production at Standard Titre

The most productive VSV G expressing cell lines and wild-type HEK 293 cells were seeded in DMEM media (10% FCS, 1% penicillin/streptomycin) into 10 cm dishes 24 hour prior to transfection so as to be at 80% confluent at the point of transfection.

At the time of transfection, DNA/PEI complexes were made up as follows:

For the VSV G line cell transfections, the following amounts of DNA/well were added to 2.5 ml of optimum per 10 cm dish in a master solution:
5 μg Lentivirus GFP vector
5 μg Rev plasmid
12 μg Gag/pol plasmid.

An amount of branched PEI equivalent to 3× the total weight of DNA was added to another 2.5p1 of optimum per well in a separate tube.

Both solutions were filtered through a 0.2 μm sterile filter, and then the PEI solution was added dropwise to the DNA solution.

The same protocol was used to make up a transfection complex for the transfection of the unmodified HEK 293 cells which also included 1.5 μg of VSV G plasmid.

The transfection complexes were incubated for 20 minutes at room temperature in which time the cells were washed with optimum media, then 5 ml of each transfection complex was added to the respective 10 cm dishes. The cells were left to incubate at 37° C., 5% CO₂ for 5 hours, after which time the media was changed for DMEM (10% FCS, 1% penicillin/streptomycin).

This virus containing supernatant was harvested at 48 hours and replaced with fresh media. The supernatant was again harvested at 72 hours post-transfection and stored at 4° C.

In order to calculate virus titre, wild type HEK 293 cells were seeded in DMEM (10% FCS, 1% penicillin/streptomycin) to a density of 90% in a 48 well plate 24 hours prior to infection. Four concentrations of harvested supernatant from each time point were used to infect cells in triplicate wells. Three five-fold serial dilutions were made into DMEM (10% FCS, 1% penicillin/streptomycin). Cells were infected by removing the overnight media, and adding 500uμl of diluted supernatant.

Cells were incubated at 37° C., 5% CO₂ for 48 hours. After 48 hours, samples were analysed on a BD FACSCalibur using an Argon 488 Laser, gated for positivity against unstained HEK 293 cells. The level of GFP positive cells was then used to calculate virus titre.

### Example 8: Measurement of Cell Viability

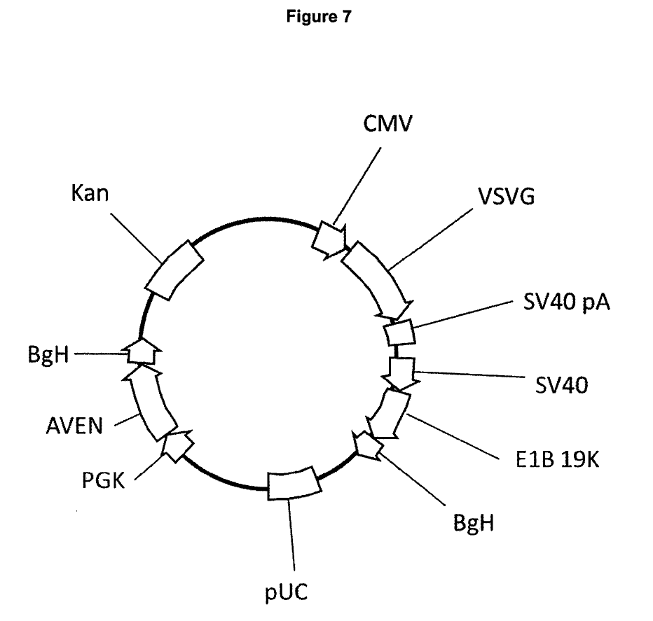

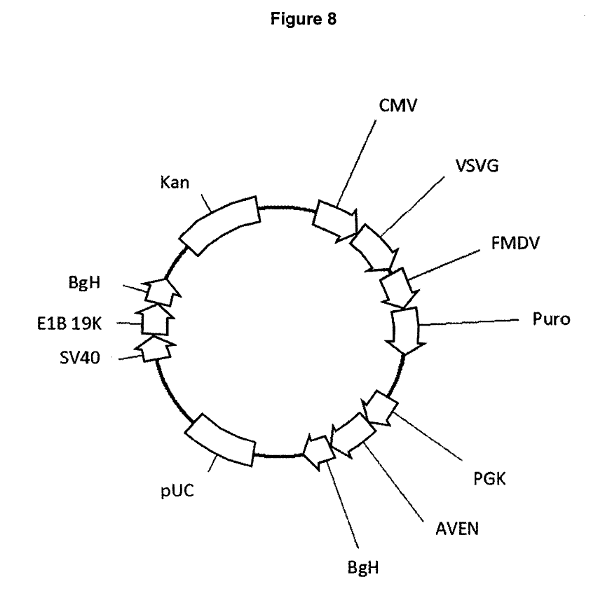

To determine the effectiveness of incorporating apoptosis inhibitors into DNA constructs, plasmids were generated encoding the VSV G glycoprotein and the puromycin resistance gene with and without a range of apoptosis inhibitor genes (e.g. FIGS. 7-8). When DNA plasmids without apoptosis inhibitors were transfected into cells, colonies were formed at 3 μg/ml and 5 μg/ml puromycin. However, compared to cells transfected with DNA constructs containing apoptosis inhibiting genes, approximately 20-fold less colonies were formed. Those colonies that did grow, particularly at 5 μg/ml puromycin, were both small, slow growing, and all colonies showed diffuse, ill-defined, membrane boundaries when growing in close proximity. Over a period of 6-8 weeks, these cells continued to grow slowly and were morphologically distinct from standard HEK 293 cells and those cell lines generated by simultaneously incorporating genes encoding apoptosis inhibitors. When sufficient cell numbers were available for lentivirus production, cell lines without apoptosis inhibitors were found to be significantly more sensitive to transfection mediated toxicity, perhaps due to the combined toxicity of both VSV G and the transfection process. These cells also produced virus at significantly lower levels compared to all of the cell lines generated by incorporating apoptosis inhibiting genes. These data demonstrate, in keeping with early observations (Pear et al., Proc. Natl. Acad. Sci. USA, Vol. 90, pp. 8392-8396, 1993) that cell lines can be generated without the addition of apoptosis inhibitors, but these cells grow poorly and have major limitations for retrovirus production.

Plasmids such as those shown in FIG. 8, where the VSV G coding region is separated from the resistance gene (e.g. for puromycin selection) by an IRES, ensuring that both are expressed in the same mRNA and thereby avoiding silencing of the toxic protein (eg. VSV G) by methylation, invariably allowed production of stable cell lines expressing higher levels of the toxic protein (e.g. VSV G).

### Example 9: Use of CMV Promoter

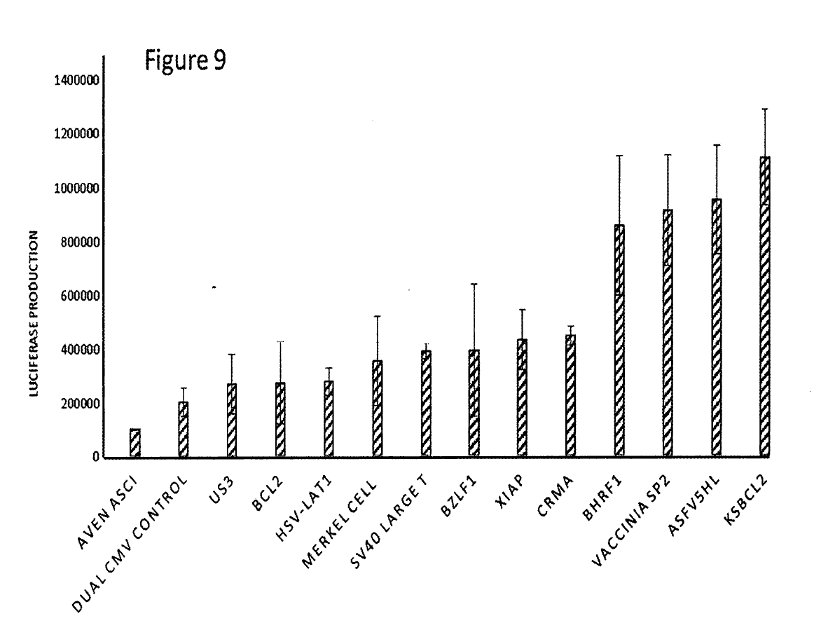

The effect of a range of individual genes encoding apoptosis inhibitors, expressed using a strong CMV promoter, on the levels of luciferase expression observed in HEK293 cells is shown in FIG. 9. Luciferase was also regulated by a CMV promoter. The most effective genes included KSbcIL2, BHRF1, Vaccinia SP2 and ASFV5HL. Interestingly, XIAP was less effective when expressed from a CMV promoter.

### Example 10: Use of SV40 Promoter

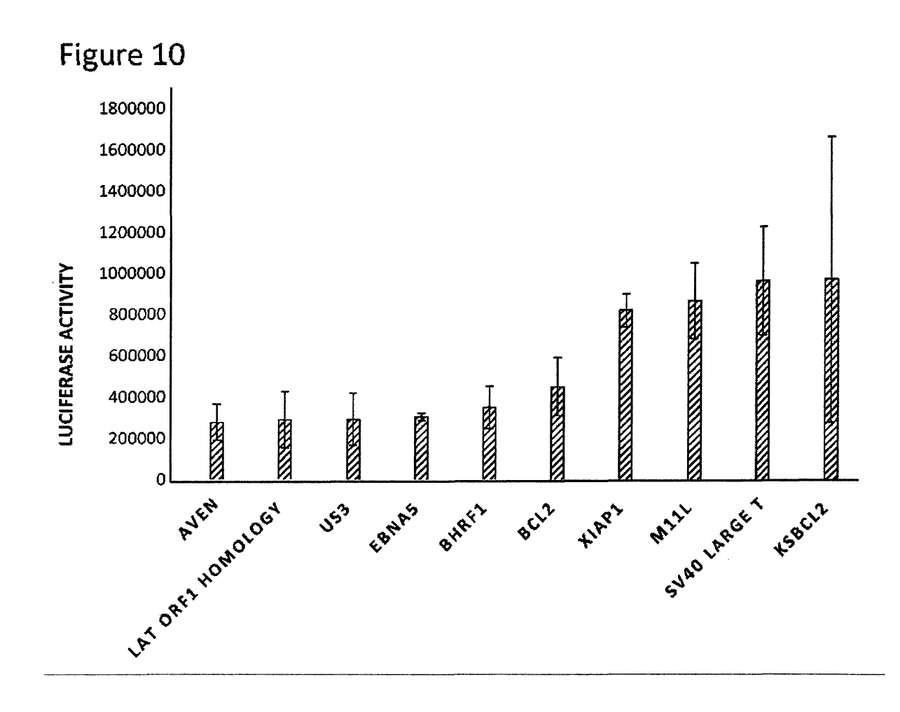

The effect of a range of individual genes encoding apoptosis inhibitors, expressed using an SV40 promoter (which is substantially weaker than a CMV promoter) on the levels of luciferase expression observed in HEK293 cells is shown in FIG. 10. Luciferase was regulated by a CMV promoter. The most effective genes included KSbcl2, XIAP, M11L and SV40 Large T antigen. BHRF1 was less effective when expressed at these relatively low levels.

### Example 11: Use of Pairs of Apoptosis Inhibitor Genes

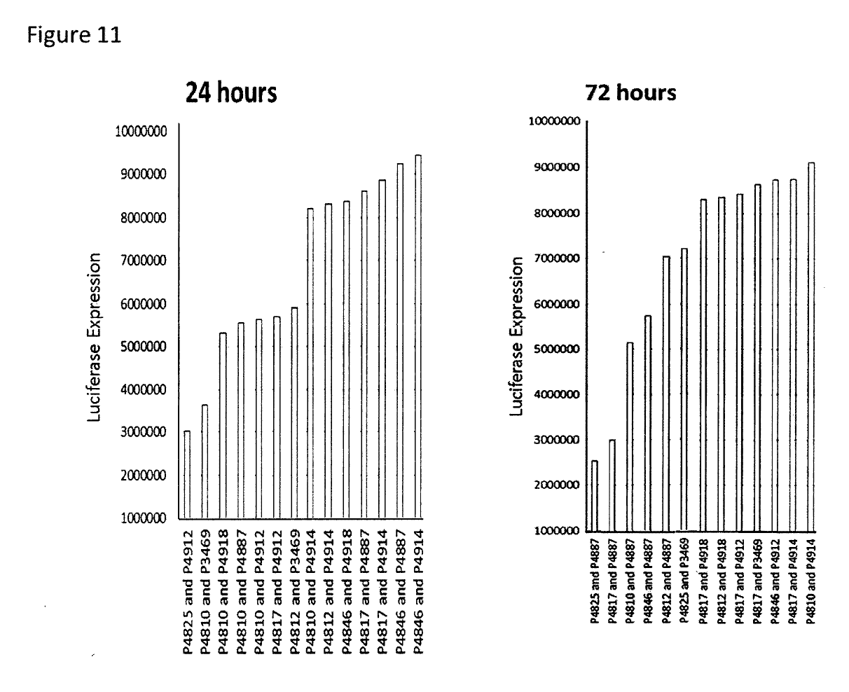

The effect of combining pairs of apoptosis inhibitor genes under the control of CMV and SV40 promoters, to give comparatively high and low levels of expression, respectively, in HEK293 cells is shown in FIG. 11. Luciferase expression was driven by the CMV promoter and was measured after 24 and 48 hours. The most effective combinations over 24 hours included highKSbc12+lowXIAP; highKSbc12+lowM11L; high BHRF1+lowKSbcl2. The most effective combinations over 48 hours included high BHRF1+lowXIAP; highASFV5HL+lowXIAP; and highKSbcl2+lowlAP1.

### Example 12: Expression of Human IgG Antibody

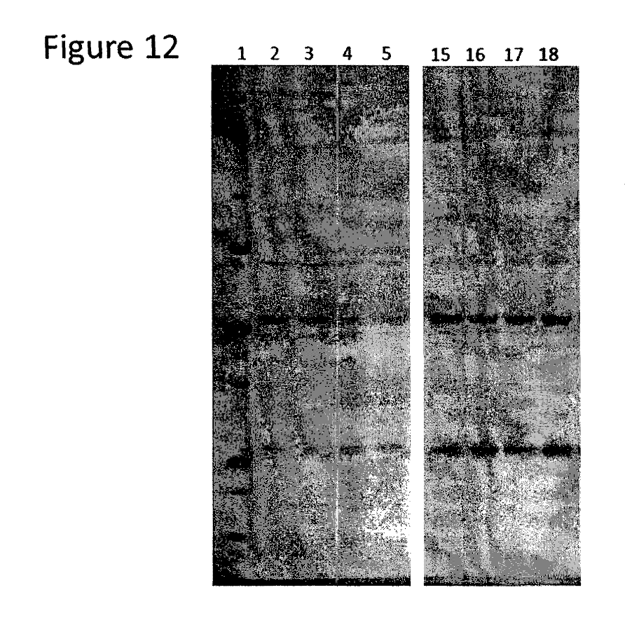

Human IgG antibody expression in suspension HEK-293 cells using a specific apoptosis inhibitor combination, namely KSbcl2 and BHRF1 both under regulatory control of the CMV promoter, is shown in FIG. 12. Lanes 1: ladder. Lanes 2&3: competitor's plasmid at 12 μg. Lanes 3&4: competitor's plasmid at 24 μg. Lanes 15&16: plasmid containing a specific apoptosis inhibitor combination at 12 μg. Lanes 17&18: plasmid containing a specific apoptosis inhibitor combination at 24 μg. Apoptosis inhibitors allow the usage of higher DNA quantities without seeing the reduction in protein expression seen in lanes 4&5.

### Example 13: Expression of Yellow Fever NS1 Secreted Protein

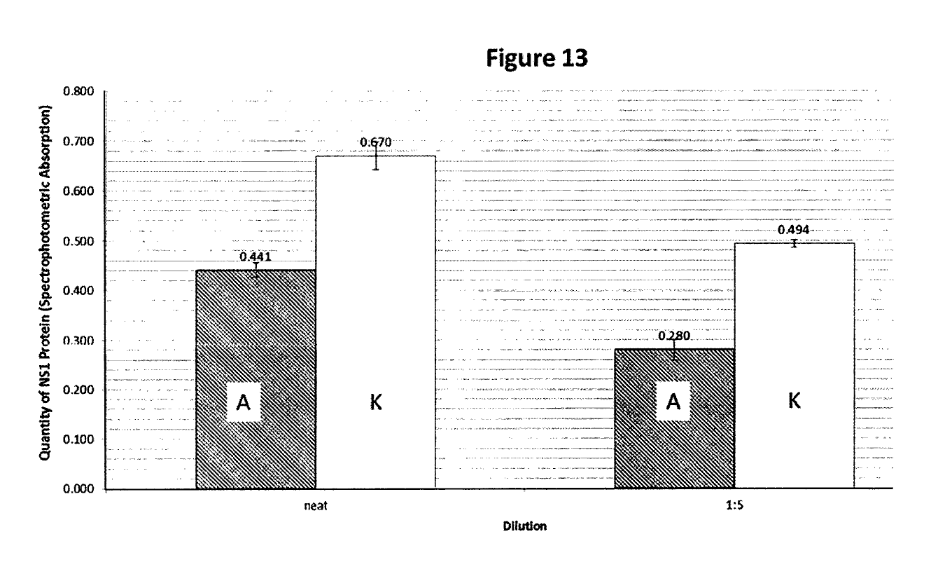

Yellow fever NS1 secreted protein expression was measured via ELISA after 3 days from transfected suspension HEK-293 cells. Sample A contains a standard CMV expression plasmid. Sample K contains a plasmid encoding KSbcl2 apoptosis inhibitor under regulatory control of the CMV promoter, alongside the CMV promoter-driven NS1 plasmid. The KSbcl2 gene significantly increased NS1 protein expression as shown in FIG. 13.

### SEQUENCES

(VSV G, Vesicular Stomatitis Virus)
SEQ ID NO: 1
MKCLLYLAFLFIGVNCKFTIVFPHNQKGNWKNVPSNYHYCPSSSDLNWHND
LIGTALQVKMPKSHKAIQADGWMCHASKWVTTCDFRWYGPKYITHSIRSFT
PSVEQCKESIEQTIKQGTWLNPGFPPQSCGYATVTDAEAVIVQVTPHHVLV
DEYTGEWVDSQFINGKCSNYCPTVHNSTTWHSDYKVKGLCDSNLISMDITF
FSEDGELSSLGKEGTGFRSNYFAYETGGKACKMQYCKHWGVRLPSGVWFEM
ADKDLFAAARFPECPEGSSISAPSQTSVDVSLIQDVERILDYSLCQETWSK
IRAGLPISPVDLSYLAPKNPGTGPAFTIINGTLKYFETRYIRVDIAAPILS
RMVGMISGTTTERELWDDWAPYEDVEIGPNGVLRTSSGYKFPLYMIGHGML
DSDLHLSSKAQVFEHPHIQDAASQLPDDESLFFGDTGLSKNPIELVEGWFS
SWKSSIASFFFIIGLIIGLFLVLRVGIHLCIKLKHTKKRQIYTDIEMNRLG
K
(GAG-POL, Human Immunodeficiency Virus)
SEQ ID NO: 2
MGARASVLSGGELDRWEKIRLRPGGKKKYKLKHIVVVASRELERFAVNPGL
LETSEGCRQILGQLQPSLQTGSEELRSLYNTVATLYCVHQRIEIKDTKEAL
DKIEEEQNKSKKKAQQAAADTGHSSQVSQNYPIVQNIQGQMVHQAISPRTL
NAWVKVVEEKAFSPEVIPMFSALSEGATPQDLNTMLNTVGGHQAAMQMLKE
TINEEAAEWDRVHPVHAGPIAPGQMREPRGSDIAGTTSTLQEQIGWMTNNP
PIPVGEIYKRWIILGLNKIVRMYSPTSILDIRQGPKEPFRDYVDRFYKTLR
AEQASQEVKNWMTETLLVQNANPDCKTILKALGPAATLEEMMTACQGVGGP
GHKARVLAEAMSQVTNSATIMMQRGNFRNQRKIVKCFNCGKEGHIARNCRA
PRKKGCWKCGKEGHQMKDCTERQANFLREDLAFLQGKAREFSSEQTRANSP
TISSEQTRANSPTRRELQVVVGRDNNSLSEAGADRQGTVSFNFPQITLWQR
PLVTIKIGGQLKEALLDTGADDTVLEEMSLPGRWKPKMIGGIGGFIKVRQY
DQILIEICGHKAIGTVLVGPTPVNIIGRNLLTQIGCTLNFPISPIETVPVK
LKPGMDGPKVKQWPLTEEKIKALVEICTEMEKEGKISKIGPENPYNTPVFA
IKKKDSTKWRKLVDFRELNKRTQDFWEVQLGIPHPAGLKKKKSVTVLDVGD
AYFSVPLDEDFRKYTAFTIPSINNETPGIRYQYNVLPQGWKGSPAIFQSSM
TKILEPFRKQNPDIVIYQYMDDLYVGSDLEIGQHRTKIEELRQHLLRWGLT
TPDKKHQKEPPFLWMGYELHPDKWTVQPIVLPEKDSWTVNDIQKLVGKLNW
ASQIYPGIKVRQLCKLLRGTKALTEVIPLTEEAELELAENREILKEPVHGV
YYDPSKDLIAEIQKQGQGQWTYQIYQEPFKNLKTGKYARTRGAHTNDVKQL
TEAVQKITTESIVIWGKTPKFKLPIQKETWETVWVTEYVVQATWIPEWEFV
NTPPLVKLWYQLEKEPIVGAETFYVDGAASRETKLGKAGYVTNRGRQKVVT
LTDTTNQKTELQAIHLALQDSGLEVNIVTDSQYALGIIQAQPDKSESELVN
QIIEQLIKKEKVYLAWVPAHKGIGGNEQVDKLVSAGIRKVLFLDGIDKAQD
EHEKYHSNWRAMASDFNLPPVVAKEIVASCDKCQLKGEAMHGQVDCSPGIW
QLDCTHLEGKVILVAVHVASGYIEAEVIPAETGQETAYFLLKLAGRWPVKT
IHTDNGSNFTSTTVKAACVWVAGIKQEFGIPYNPQSQGVVESMNKELKKII
GQVRDQAEHLKTAVQMAVFIHNFKRKGGIGGYSAGERIVDIIATDIQTKEL
QKQITKIQNFRVYYRDSRDPLWKGPAKLLWKGEGAVVIQDNSDIKVVPRRK
AKIIRDYGKQMAGDDCVASRQDED

1. A process for producing a mammalian cell which constitutively expresses one or more cytotoxic virus polypeptides, the process comprising the steps:
(i) introducing one or more nucleic acid molecules encoding
(a) one or more cytotoxic virus polypeptides, and
(b) one or more apoptosis inhibitors into a mammalian cell; and
(ii) culturing the cell under conditions such that the one or more cytotoxic virus polypeptides and the one or more apoptosis inhibitors are constitutively expressed,
wherein the expression of the one or more apoptosis inhibitors prevents apoptosis of the cell, and wherein the one or more nucleic acid molecules additionally comprises a selection gene which is located in the same primary transcript as the nucleic acid encoding the one or more cytotoxic virus polypeptides and is transcribed in the same mRNA molecule.

2. A process as claimed in claim 1, where the selection gene is inserted after an internal ribosome entry site (IRES) downstream of the stop codon of the nucleic acid encoding the one or more cytotoxic virus polypeptides.

3. A process as claimed in claim 1 or claim 2, wherein one or more of the cytotoxic virus polypeptides are selected from the group consisting of VSV G and Gag-Pol.

4. A process as claimed in any one of claims 1 to 3, wherein the mammalian cell is a human cell.

5. A process as claimed in any one of claims 1 to 4, wherein the mammalian cell is an immortalised cell, preferably a HEK 293 cell.

6. A process as claimed in any one of the preceding claims, wherein one or more of the apoptosis inhibitors is a polypeptide or RNA.

7. A process as claimed in any one of the preceding claims, wherein one or more of the apoptosis inhibitors is an inhibitor of the APAF-1, Caspase 9, BAK, BAX or BAD pathway.

8. A process as claimed in any one of the preceding claims, wherein the apoptosis inhibitors inhibit more than one of the APAF-1, Caspase 9, BAK, BAX or BAD pathways.

9. A process as claimed in any one of the preceding claims, wherein one or more of the apoptosis inhibitors is Celovirus GAM1, Adenovirus E4 Orf6, Adenovirus E1B 55K, Adenovirus FIB 19K, Myxomavirus M11L, Cytomegalovirus 1E1, Cytomegalovirus 1E2, Baculovirus p35, Baculovirus IAP-1, Herpesvirus US3, Herpesvirus Saimiri ORF16, Herpes Simplex 2 LAT ORF 1, Human XIAP, African Swine Fever ASFV-5-HL (LMW-5-HL/A179L), Kaposi's Sarcoma virus KSbcl2, Vaccinia virus SPI-2, Cowpoxvirus CrmA, Epstein Barr virus BHRF1, Epstein Barr virus EBNA-5, Epstein Barr virus BZLF-1, Papillomavirus E6, Human Aven, Human BCL2 or Human BCL-XL.

10. A process as claimed in claim 9, wherein one or more of the apoptosis inhibitors is AVEN or E1B-19K.

11. A process as claimed in any one of the preceding claims, wherein the one or more nucleic acid molecules encode two apoptosis inhibitor polypeptides.

12. A process as claimed in claim 11, wherein the two apoptosis inhibitors are AVEN and Adenovirus serotype 5 E1B-19K.

13. A process as claimed in claim 9, wherein the one or more nucleic acid molecules encode one, two or three apoptosis inhibitor polypeptides selected from the group consisting of Human XIAP, Kaposi's Sarcoma virus KSbcl2 and Epstein Barr virus BHRF1.

14. A process as claimed in claim 9, wherein the one or more nucleic acid molecules encode BHFR1 and also one or more apoptosis inhibitor polypeptides selected from the group consisting of BCL-XL, ASFV-5-HL and Vaccinina virus SPI-2.

15. A process as claimed in any one of the preceding claims, wherein the expression of the one or more cytotoxic virus polypeptides and/or the expression of the one or more apoptosis inhibitors is driven by one or more constitutive promoters.

16. A process as claimed in claim 15, wherein the one or more constitutive promoters are selected from the group consisting of the CMV, SV40, PGK (human or mouse), HSV TK, SFFV, Ubiquitin, Elongation Factor Alpha, CHEF-1, FerH, Grp78, RSV, Adenovirus EIA, CAG and CMV-Beta-Globin promoters, preferably from the group consisting of the RSV, CMV, SV40, PGK and ubiquitin promoters.

17. A process as claimed in claim 15, wherein the apoptosis inhibitors are human Aven and Adenovirus serotype 5 E1B-19K, and wherein the expression of human Aven and Adenovirus serotype 5 E1B-19K is driven by different promoters selected from RSV, CMV, SV40, PGK and ubiquitin promoters.

18. A mammalian cell which is obtained or obtainable by a process as claimed in any one of the preceding claims.

19. A cell line wherein the cells of the cell line constitutively express one or more cytotoxic virus polypeptides and one or more apoptosis inhibitors, wherein the expression of the apoptosis inhibitor(s) prevents apoptosis of the cells of the cell line, and wherein the cells of the cell line additionally express a selection gene product which is transcribed in the same mRNA molecule as the one or more cytotoxic virus polypeptides.

20. A cell line as claimed in claim 19, where the selection gene is inserted after an internal ribosome entry site downstream of the stop codon of the nucleic acid encoding the one or more cytotoxic virus polypeptides.

21. A cell line as claimed in claim 19 or claim 20, wherein one or more of the cytotoxic virus polypeptides are selected from the group consisting of VSV G and Gag-Pol.

22. A cell line as claimed in any one of claims 19 to 21, wherein one or more of the apoptosis inhibitors is Celovirus GAM1, Adenovirus E4 Orf6, Adenovirus E1B 55K, Adenovirus FIB 19K, Myxomavirus M11L, Cytomegalovirus 1E1, Cytomegalovirus 1E2, Baculovirus p35, Baculovirus IAP-1, Herpesvirus US3, Herpesvirus Saimairi ORF16, Herpes Simplex 2 LAT ORF 1, Human XIAP, African Swine Fever ASFV-5-HL (LMW-5-HL/A179L), Kaposi's Sarcoma virus KSbcl2, Vaccinia virus SPI-2, Cowpoxvirus CrmA, Epstein Barr virus BHRF1, Epstein Barr virus EBNA-5, Epstein Barr virus BZLF-1, Papillomavirus E6, Human Aven, Human BCL2 or Human BCL-XL, preferably AVEN or E1B-19K.

23. A cell line as claimed in claim 22, wherein the cells of the cell line constitutively express one, two or three apoptosis inhibitor polypeptides selected from the group consisting of Human XIAP, Kaposi's Sarcoma virus KSbcl2 and Epstein Barr virus BHRF1.

24. A cell line as claimed in claim 22, wherein the cells of the cell line constitutively express BHFR1 and also one or more apoptosis inhibitor polypeptides selected from the group consisting of BCL-XL, ASFV-5-HL and Vaccinia virus SPI-2.

25. A cell line as claimed in any one of claims 19 to 24, wherein the cell line is one which is capable of being passaged at least 5×, more preferably at least 10× and most preferably at least 15×.

26. A nucleic acid molecule encoding one or more cytotoxic virus polypeptides and one or more apoptosis inhibitors, wherein the nucleic acid molecule additionally comprises a selection gene which is located in the same primary transcript as the nucleic acid encoding the one or more cytotoxic virus polypeptides and is transcribed in the same mRNA molecule.

27. A nucleic acid molecule as claimed in claim 26, where the selection gene is inserted after an internal ribosome entry site downstream of the stop codon of the nucleic acid encoding the one or more cytotoxic virus polypeptides.

28. A nucleic acid molecule as claimed in claim 26 or claim 27, wherein one or more of the cytotoxic virus polypeptides are selected from the group consisting of VSV G and Gag-Pol.

29. A nucleic acid molecule as claimed in any one of claims 26 to 28, wherein one or more of the apoptosis inhibitors is Celovirus GAM1, Adenovirus E4 Orf6, Adenovirus E1B 55K, Adenovirus E1B 19K, Myxomavirus M11L, Cytomegalovirus 1E1, Cytomegalovirus 1E2, Baculovirus p35, Baculovirus IAP-1, Herpesvirus US3, Herpesvirus Saimairi ORF16, Herpes Simplex 2 LAT ORF 1, Human XIAP, African Swine Fever ASFV-5-HL (LMW-5-HL/A179L), Kaposi's Sarcoma virus KSbcl2, Vaccinia virus SPI-2, Cowpoxvirus CrmA, Epstein Barr virus BHRF1, Epstein Barr virus EBNA-5, Epstein Barr virus BZLF-1, Papillomavirus E6, Human Aven, Human BCL2 or Human BCL-XL, preferably AVEN or E1B-19K.

30. A nucleic acid molecule as claimed in any one of claims 26 to 29, wherein the nucleic acid molecule encodes one, two or three apoptosis inhibitor polypeptides selected from the group consisting of Human XIAP, Kaposi's Sarcoma virus KSbcl2 and Epstein Barr virus BHRF1.

31. A nucleic acid molecule as claimed in any one of claims 26 to 29, wherein the nucleic acid molecule encodes BHFR1 and also one or more apoptosis inhibitor polypeptides selected from the group consisting of BCL-XL, ASFV-5-HL and Vaccinina virus SPI-2.

32. A kit comprising:
(i) a nucleic acid molecule encoding one or more cytotoxic virus polypeptides and a selection gene, wherein the selection gene is located in the same primary transcript as the nucleic acid molecule encoding the one or more cytotoxic virus polypeptides and is transcribed in the same mRNA molecule, and
(ii) a nucleic acid molecule encoding one or more apoptosis inhibitors,
for use in simultaneously, sequentially or separately introducing the nucleic acid molecules into a mammalian cell, in order to produce cells which constitutively express the one or more cytotoxic virus polypeptides and the one or more apoptosis inhibitors, and the selection gene product and wherein the expression of the apoptosis inhibitor(s) prevents apoptosis of the cells.

33. A process for producing retroviruses, the process comprising the steps:
(a) introducing one or more nucleic acids encoding a retrovirus into a cell as defined in claim 18 or into a cell of a cell line as defined in any one of claims 19 to 25;
(b) introducing one or more helper plasmids encoding one or more of Rev, Gag-Pol, or Tat polypeptides into the cell;
(c) culturing the cell under conditions such that retroviruses are assembled and secreted by the cell; and
(d) harvesting packaged retrovirus.

34. A process as claimed in claim 33, wherein the retrovirus is a lentivirus.

35. A process as claimed in claim 33 or claim 34, wherein two helper plasmids are used, wherein the first encodes Gag-Pol and the second encodes Rev.

36. A process for producing a recombinant polypeptide, the process comprising the steps:
(a) producing retroviruses by a process as defined in any one of claims 33 to 35, wherein the retroviral genomes comprise a nucleic acid molecule encoding a desired recombinant polypeptide;
(b) introducing the retroviruses into a host cell;
(c) culturing the host cells under conditions such that the desired recombinant polypeptide is produced; and
(d) harvesting the desired polypeptide.
